(19) United States
(12) Patent Application Publication
(10) Pub. No.: US 2026/0041454 A1
(43) Pub. Date: Feb. 12, 2026

(54) HANDS-FREE DISPOSABLE CANNULA SYSTEMS AND DEVICES PROVIDING 3D IMAGES FOR ARTHROSCOPIC AND ENDOSCOPIC PROCEDURES

(71) Applicant: Earendel Medical Ltd., Gedera (IL)

(72) Inventors: Guy Levy, Gedera (IL); Chen Levin, Ramat Gan (IL); Moran Shochat, Zichron Yaakov (IL); Nir Lilach, Kfar Yehoshua (IL); Amit Segal, Afula (IL)

(21) Appl. No.: 19/292,974

(22) Filed: Aug. 7, 2025

Related U.S. Application Data
(60) Provisional application No. 63/680,755, filed on Aug. 8, 2024.
(60) Provisional application No. 63/792,348, filed on Apr. 22, 2025.

(51) Int. Cl. A61B 17/34; A61B 1/00; A61B 1/313; A61B 1/317

(52) U.S. Cl. CPC A61B 17/3423; A61B 1/00045; A61B 1/3132; A61B 1/317

(57) ABSTRACT

A cannula system is provided that enables hands-free operation of cameras by a surgeon and provides 3D images of the surgical site. An exemplary cannula system comprises a cannula having a proximal end, a distal end, a central lumen, and an insertion axis. A disc is mounted to the distal end of the cannula. The disc has two separable half portions, and each separable half portion defines at least one internal slot. A camera is housed in each of the separable half portions in the internal slots. Each camera is aligned with the insertion axis and live streams during insertion of the cannula system to provide under vision insertion. An obturator is configured to be at least partially disposed within the central lumen of the cannula. When the obturator is removed the two separable half portions of the disc can be slid apart to surgical position.

### CROSS-REFERENCE TO RELATED APPLICATIONS

This application is a non-provisional of and claims priority to U.S. Patent Application Ser. No. 63/792,348, filed Apr. 22, 2025, and U.S. Patent Application Ser. No. 63/680,755, filed Aug. 8, 2024, each of which is hereby incorporated by reference in its entirety.

### FIELD OF THE DISCLOSURE

The following disclosure relates to cannula systems and devices for arthroscopic and endoscopic procedures.

### BACKGROUND

Arthroscopy is a type of keyhole surgery for checking or repairing a patient's joints. In keyhole surgery, only small cuts are made into the body. Laparoscopy is a surgical procedure used to examine the organs in the abdomen. It can also be used to examine a woman's pelvic organs.

In the 1960s, optic glass fibers were invented and used in the arthroscopic line of Karl Storz. Over the years, there have been advances in instruments for these surgical procedures, including improvements in technology, camera weight, synchronization and resolution quality. Wireless cameras and nano cameras have been introduced.

However, significant drawbacks remain in arthroscopic and laparoscopic surgical instruments or integrated robotics systems and robotics instruments. For instance, typically they do not allow the surgeon to view the surgical area in 3D. Also, current endoscopes require that the surgeon hold the camera instrument in one hand.

Accordingly, there is a need for improved arthroscopic and laparoscopic surgical instruments that facilitate 3D imaging and better vision for surgeons. There is also a need for improved endoscopes that allow for hands-free operation of the cameras.

### SUMMARY

The present disclosure, in its many embodiments, alleviates to a great extent the disadvantages of known cannula systems for arthroscopic and laparoscopic surgical procedures by providing cannula systems with several different novel mechanisms for deploying cameras to the surgical site. In exemplary embodiments, cannula systems have movable positioning members, each housing a camera so the multiple cameras align with the insertion axis of the cannula and provide three-dimensional images of the surgical site. Exemplary cannula systems may have a single cannula or both an inner and outer cannula.

The present disclosure also provides cannula systems with movable flaps housing cameras. The flaps are movable from a closed position to an open position in which the cameras align with the insertion axis of the cannula. Embodiments of the disclosure include cannula systems with a disc housing cameras aligned with the insertion axis of the cannula, providing under vision insertion and 3D imaging for surgery. In exemplary embodiments, cameras are housed in the distal end of the cannula so they are aligned with the insertion axis. Embodiments of the cannula system enable hands-free operation of the cameras because the integration of the cannula and cameras allows the surgeon to operate the surgical tools freely via the cannulas, obviating the need to independently hold the cameras. In exemplary embodiments, the cannula is disposable. Disclosed cannula systems provide both static/fixed camera placement and movable positioning camera deployment.

As used herein, the terms “proximal” and “distal” are defined in relation to a surgeon (or other medical practitioner) and a patient. The term “proximal” refers to the position of an element arranged closer to the surgeon and further away from the patient, and the term “distal” refers to the position of an element arranged further away from the surgeon and closer to the patient.

Exemplary embodiments include a disposable cannula having a working channel with a plurality of cameras, which may be attached to the distal side. Exemplary devices may include one or more Light Emitting Diodes (LEDs) and tracks for gas/water/suction. The cameras can be wireless or connected, and may be seated in the distal part of the cannula. The cameras may feature internal movement tracking capability to allow tracking of the movement of the instruments and to provide the ability to generate 3D images.

A separate part of the system may be connected to the surgical instruments. This device may be a pre-connected camera instrument that includes one or more cameras. An image sensor can be folded and able to be opened by the surgeon. This part may be detachable and can be hooked on as needed.

The command center of the system is reusable and includes software and/or hardware, which controls the camera and LED operation and the synchronization among the cameras. It also allows the right connection between the system and the operating room facilities like water, gas and suction. It can involve AI or other software to choose the right image and other features like bleeding control.

Exemplary embodiments advantageously provide under vision insertion of a cannula. A cannula system comprises at least one cannula, a disc mounted to the distal end of the cannula, a camera, and an obturator. The cannula has a proximal end, a distal end, a central lumen, and an insertion axis. The disc has two separable half portions, and a camera and one or more LED modules are housed in each of the separable half portions of the disc so that each camera is aligned with the insertion axis. In exemplary embodiments, the disc defines a channel configured to house one or more wires. The obturator is configured to be at least partially disposed within the central lumen of the cannula and may have a cutting edge at or near its distal end.

In exemplary embodiments, the obturator is substantially flat and has a cone-shaped distal end with a cutting edge. The cone-shaped distal end may define at least one channel configured to be aligned with each camera so each camera has an unobstructed view of the surgical site along the insertion axis. When the obturator is removed, the two separable half portions of the disc can be slid apart by the surgeon using one or more handles.

In exemplary embodiments, each separable half portion of the disc has a flange defining at least one internal slot, and each internal slot houses a camera and one or more LED modules. In exemplary embodiments, each camera starts streaming live video prior to the insertion of the cannula system, providing the surgeon visual coverage of the cannula insertion. The cannula system may further comprise a processor in communication with the camera. The processor receives images from the camera and provides the images in three dimensions. In exemplary embodiments, the cannula system further comprises at least one irrigation connector.

Exemplary methods of providing under vision insertion and 3D imaging for surgery comprise the steps of providing at least one cannula, inserting the cannula and an obturator partially disposed within the central lumen of the cannula into a surgical joint or cavity space, and removing the obturator from the cannula. The cannula has a proximal end, a distal end, an insertion axis, and a disc mounted to its distal end. The disc comprises two separable half portions, and a camera is housed in each of the separable half portions of the disc. Each camera is aligned with the insertion axis and is active during insertion. Methods include the step of sliding the two separable half portions of the disc apart using one or more handles. One or more irrigation connectors may be attached to the cannula. A processor may be provided to be in communication with each camera.

In exemplary embodiments, the cannula system comprises a cannula, an obturator, a camera module, and a ring. The cannula has a proximal end and a distal end, a central lumen, a ring, and a plurality of movable arms near the distal end. Each of the plurality of movable arms houses a camera. The camera module is configured to be at least partially disposed within the cannula. A processor is in communication with the cameras.

Exemplary cannula systems and methods comprising a cannula having a proximal end, a distal end, a central lumen, and an insertion axis. An obturator is configured to be at least partially disposed within the central lumen of the cannula, and the system includes at least one positioning member and a camera mounted to the at least one positioning member. In exemplary embodiments, the positioning member is a strip positioner. The cannula may have one or more slots configured to receive positioning members when they are deployed. The obturator may have one or more slots to allow the cameras unobstructed views of the surgical site. A method of providing 3D imaging for surgery comprises inserting a cannula and an obturator partially disposed within the cannula into a surgical joint or cavity space, withdrawing the obturator from the cannula, inserting at least one positioning member with a camera mounted thereto through the cannula and into the joint or cavity space, and activating the camera.

Exemplary embodiments of a cannula system comprise an outer cannula and an inner cannula. The outer cannula has a proximal end, a distal end, a central lumen, and an insertion axis. The inner cannula has a proximal end, a distal end, a central lumen, and an insertion axis and is configured to be at least partially disposed within the outer cannula. The distal end of the outer cannula forms a disc and has at least one camera housed in the disc aligned with the insertion axis. In exemplary embodiments, the cameras may be positioned at about nine o'clock and about three o'clock of the disc. For certain applications, such as shoulder surgery, the cameras may be positioned at about 12 o'clock and about 6 o'clock of the disc.

In another exemplary embodiment, a cannula system comprises at least one cannula, an obturator, at least one positioning member, and a camera. The cannula has a proximal end, a distal end, a central lumen, and an insertion axis. The obturator has a proximal end, a distal end, and an insertion axis and is configured to be at least partially disposed within the cannula. The positioning member has a proximal end, a distal end, and a flexible portion near the distal end, and a camera is located at or near the distal end of the positioning member. When the distal end of the at least one positioning member exits the distal end of the at least one cannula the flexible portion bends such that the camera is aligned with the insertion axis.

In exemplary embodiments, the cannula system further comprises an inner cannula having a proximal end and a distal end. The inner cannula is configured to be deployed within the cannula along the insertion axis after the obturator is removed. The positioning member may be integrated with the distal end of the inner cannula. In exemplary embodiments, the inner cannula defines one or more lumens configured for passage of fluids or suction. The cannula system may further comprise a ring at or near the distal end of the inner cannula and a camera housed in the ring. In an exemplary embodiment, the cannula opens at the joint by passing through a ring made of silicone or other materials.

The cannula system may further comprise a processor in communication with the camera. The processor receives images from the camera and provides the images in three dimensions. In exemplary embodiments, the obturator has a cutting edge at or near its distal end. The obturator also may be equipped with an ergonomic handle and/or a locking mechanism at or near its proximal end. The cannula system may be configured for hands-free operation of the cameras and may be disposable. In exemplary embodiments, the cannula system further comprises at least one irrigation connector at or near the proximal end of the cannula.

In exemplary embodiments, the positioning member is a strip positioner. The positioning member may be made of nitinol. The obturator may have at least one slot configured to receive the camera, and the cannula may have at least one slot configured to receive the at least one positioning member.

An exemplary method of providing 3D imaging for surgery comprises inserting at least one cannula and an obturator partially disposed within the cannula into a surgical joint or cavity space, withdrawing the obturator from the cannula, and inserting at least one positioning member through the central lumen of the cannula along the insertion axis and into the joint or cavity space. The cannula has a proximal end, a distal end, and an insertion axis. The positioning member has a proximal end, a distal end, and a flexible portion near the distal end of the positioning member. At least one camera is located at or near the distal end of the positioning member. When the distal end of the positioning member exits the distal end of the at least one cannula, the flexible portion bends so the camera is aligned with the insertion axis.

Methods may further comprise pre-connecting the camera and the positioning member and activating the camera. Alternatively, a live camera can be introduced during placement of the cannula. Exemplary methods further comprise the step of retracting the positioning member and removing the cannula after completion of a surgical procedure.

A cannula system with movable flaps also is provided. An exemplary embodiment of such a cannula system comprises at least one cannula, an obturator, and at least one camera. Embodiments could have a single cannula or both an outer cannula and an inner cannula. The single cannula (or outer cannula) has a proximal end, a distal end, a central lumen, an insertion axis, and flaps at the distal end. A camera is housed in each of the flaps, which are movable between a closed position and an open position. In exemplary embodiments, an inner cannula is configured to be at least partially disposed within the central lumen of the outer cannula along the insertion axis, and the obturator is configured to be at least partially disposed within the inner cannula along the insertion axis.

When in position within the inner cannula, the obturator holds the flaps in the closed position and serves to protect the cameras, keeping them covered during deployment into the surgical site. When the obturator is removed and the inner cannula is pushed distally, the inner cannula moves the flaps into the open position such that each camera is activated and aligned with the insertion axis.

Exemplary embodiments further comprise at least one irrigation connector at or near the proximal end of the cannula. The cannula system may further comprise a processor in communication with the camera. The processor receives images from the camera and provides the images in three dimensions. The obturator may have a cutting edge at or near its distal end.

An exemplary method of providing 3D imaging for surgery includes the steps of providing an outer cannula, an inner cannula, and an obturator. The outer cannula has a proximal end, a distal end, an insertion axis, and flaps at the distal end. Each of the flaps houses a camera and is movable between a closed position and an open position. The inner cannula has a proximal end, a distal end, and an insertion axis, and the obturator is configured to hold the flaps in the closed position.

In exemplary embodiments, the inner cannula is inserted into the central lumen of the outer cannula, and the obturator is inserted into the inner cannula. Then the full cannula system is inserted into the surgical site. Alternatively, the outer cannula is inserted into a surgical joint or cavity space. The inner cannula is inserted into the outer cannula and the obturator is inserted into the inner cannula so it is at least partially disposed within the inner cannula along the insertion axis and so the obturator maintains the flaps in the closed position.

Method steps further comprise withdrawing the obturator from the central lumen of the inner cannula, then pushing the inner cannula distally to move each of the flaps into the open position so each camera is aligned with the insertion axis. The flaps are maintained in the open position for the surgical procedure.

In exemplary embodiments, cannula systems provide static or fixed placement of the cameras. Such systems comprise a cannula, an obturator, and one or more cameras. The cannula has a proximal end, a distal end, a body with a central lumen, a rear part, and an insertion axis. The obturator has a proximal end and a distal end and is configured to be disposed within the central lumen of the cannula along the insertion axis. One or more cameras are housed in the distal end of the cannula so they are aligned with the insertion axis. The rear part of the cannula is configured to house wires or circuitry. The body of the cannula may be a threaded component. In exemplary embodiments, the obturator is a rigid component with a cutting edge. The obturator may have an ergonomic handle and a locking mechanism.

The cannula may define one or more inner lumens and openings at or near the distal end for passage of gas, water, and/or suction. In exemplary embodiments, the cannula defines one or more inner lumens configured to nouse wires to pass electricity. The wires are connected to the cameras and may also be connected to one or more sensors and lights located at or near the distal end of the cannula. Valves may be provided to prevent gas or water leakage and may be integrated into the rear part of the cannula. Exemplary embodiments may further comprise one or more gimbal motors for the cameras.

Accordingly, it is seen that improved cannula systems and methods of providing 3D imaging for surgical procedures are provided. These and other features and advantages will be appreciated from review of the following detailed description, along with the accompanying figures in which like reference numbers refer to like parts throughout.

### BRIEF DESCRIPTION OF THE DRAWINGS

The above-mentioned features and objects of the present disclosure will become more apparent with reference to the following description taken in conjunction with the accompanying drawings wherein like reference numerals denote like elements and in which:

FIG. 1 is a perspective view of an exemplary embodiment of a cannula system in accordance with the present disclosure;

FIG. 2 is a perspective view of an exemplary embodiment of a cannula system in accordance with the present disclosure;

FIG. 3 is a perspective view of an exemplary embodiment of a cannula system in accordance with the present disclosure;

FIG. 4A is a perspective view of an exemplary embodiment of an outer cannula in accordance with the present disclosure;

FIG. 4B is a side view of the outer cannula of FIG. 4A;

FIG. 4C is a side cross-sectional view of the outer cannula of FIG. 4A;

FIG. 4D is a rear view of the outer cannula of FIG. 4A;

FIG. 5A is a perspective view of an exemplary embodiment of an inner cannula in accordance with the present disclosure showing positioning members in a closed position;

FIG. 5B is a perspective view of the inner cannula of FIG. 5A showing positioning members in an open position;

FIG. 5C is a side view of the inner cannula of FIG. 5A;

FIG. 5D is a side cross-sectional view of the inner cannula of FIG. 5A;

FIG. 5E is a front view of the inner cannula of FIG. 5A;

FIG. 6 is a perspective view of an exemplary embodiment of an obturator in accordance with the present disclosure;

FIG. 7A is a perspective view of an exemplary embodiment of a cannula system in closed position prior to deployment in accordance with the present disclosure;

FIG. 7B is a perspective view of the cannula system of FIG. 7A in open position and deployed;

FIG. 8A is a front view of an exemplary embodiment of a ring attachment in accordance with the present disclosure;

FIG. 8B is a perspective view of an exemplary embodiment of a camera instrument with the ring attachment of FIG. 8A;

FIG. 8C is a perspective view of an exemplary embodiment of a surgical instrument using the ring attachment of FIG. 8A;

FIG. 9A is a perspective view of an exemplary embodiment of a camera instrument in accordance with the present disclosure with the camera flap closed;

FIG. 9B is a perspective view of the camera instrument of FIG. 9A with the camera flap open;

FIG. 9C is a detail view of the camera instrument of FIG. 9A;

FIG. 9D is a side cross-sectional view of the camera instrument of FIG. 9A;

FIG. 9E is a front view of the camera instrument of FIG. 9A;

FIG. 9F is a side view of the camera instrument of FIG. 9A attached to a surgical instrument;

FIG. 10 is a perspective view of an exemplary embodiment of a cannula system with positioning members in accordance with the present disclosure;

FIG. 11 is a detail perspective view of the cannula system of FIG. 10 with the obturator removed;

FIG. 12A is a perspective view of the cannula system of FIG. 10 with positioning members deployed;

FIG. 12B is a side view of the cannula system of FIG. 10 with positioning members deployed;

FIG. 13A is a perspective view of the cannula system of FIG. 10 with positioning members deployed showing a flexible disc protecting the positioning members;

FIG. 13B is a side view of the cannula system of FIG. 10 with positioning members deployed showing a flexible disc protecting the positioning members;

FIG. 14A is a side view of an exemplary embodiment of a cannula system with a movable disc in accordance with the present disclosure;

FIG. 14B is a front view of the cannula system of FIG. 14A;

FIG. 14C is a rear view of the cannula system of FIG. 14A;

FIG. 15 is a perspective view of the cannula system of FIG. 14A being deployed;

FIG. 16 is a perspective view of the cannula system of FIG. 14A being deployed with the obturator removed;

FIG. 17 is a perspective view of the cannula system of FIG. 14A being deployed with the separable portions of the movable disc slid apart;

FIG. 18A is a front view of an exemplary embodiment of the separable portions of the movable disc slid apart in accordance with the present disclosure;

FIG. 18B is a rear view of the separable portions of the movable disc of FIG. 18A;

FIG. 19 is a side view of an exemplary embodiment of a cannula system with flaps in accordance with the present disclosure;

FIG. 20 is a side view of the cannula system of FIG. 19 with the flaps in open position;

FIG. 21 is a front view of the cannula system of FIG. 19 with the flaps in open position;

FIG. 22A is a perspective view of an exemplary embodiment of a cannula system with flaps in operation in accordance with the present disclosure;

FIG. 22B is a perspective view of the cannula system of FIG. 22A showing the obturator being removed;

FIG. 22C is a perspective view of the cannula system of FIG. 22A showing the obturator removed;

FIG. 22D is a perspective view of the cannula system of FIG. 22A showing the inner cannula pushing open the flaps;

FIG. 23A is a perspective view of an exemplary embodiment of a cannula system with forward-facing cameras housed in a disc in accordance with the present disclosure;

FIG. 23B is a side view of the cannula system of FIG. 23A;

FIG. 23C is a front view of the cannula system of FIG. 23A;

FIG. 23D is a perspective view of an exemplary outer cannula of the cannula system of FIG. 23A;

FIG. 24A is a perspective view of an exemplary embodiment of a cannula system with static cameras in accordance with the present disclosure;

FIG. 24B is a side view of the cannula system of FIG. 24A;

FIG. 24C is a front view of the cannula system of FIG. 24A;

FIG. 24D is a side cross-sectional view of the cannula system of FIG. 24A;

FIG. 24E is a side cross-sectional view of an exemplary embodiment of a cannula system with static cameras in accordance with the present disclosure;

FIG. 25 is a perspective view of an exemplary embodiment of a cannula system with static cameras in accordance with the present disclosure;

FIG. 26 is a perspective view of an exemplary embodiment of a cannula for a cannula system with static cameras in accordance with the present disclosure;

FIG. 27 is a front perspective view of an exemplary embodiment of a cannula for a cannula system with static cameras in accordance with the present disclosure;

FIG. 28A is a front perspective view of an exemplary embodiment of a cannula for a cannula system with static cameras in accordance with the present disclosure showing insertion of the cameras;

FIG. 28B is a front perspective detail view of an exemplary embodiment of a cannula for a cannula system with static cameras in accordance with the present disclosure showing insertion of the cameras;

FIG. 29A is a perspective view of an exemplary rear portion of a cannula for a cannula system with static cameras in accordance with the present disclosure;

FIG. 29B is a rear view of an exemplary rear portion of a cannula for a cannula system with static cameras in accordance with the present disclosure;

FIG. 30 is a perspective view of an exemplary obturator and cannula for a cannula system with static cameras in accordance with the present disclosure;

FIG. 31A is a rear perspective view of a rear portion of an exemplary cannula for a cannula system with static cameras in accordance with the present disclosure showing exemplary wiring;

FIG. 31B is a rear perspective view of a rear portion of an exemplary cannula for a cannula system with static cameras in accordance with the present disclosure showing a wiring cover;

FIG. 32A is a rear perspective view of a rear portion of an exemplary cannula for a cannula system with static cameras in accordance with the present disclosure showing an exemplary valve;

FIG. 32B is a rear perspective view of a rear portion of an exemplary cannula for a cannula system with static cameras in accordance with the present disclosure showing a valve cover;

FIG. 33A is a rear perspective view of a rear portion of an exemplary cannula for a cannula system with static cameras in accordance with the present disclosure showing an exemplary valve seal;

FIG. 33B is a rear perspective view of a rear portion of an exemplary cannula for a cannula system with static cameras in accordance with the present disclosure showing a valve seal cover; and

FIG. 34 is a block diagram showing an exemplary embodiment of the internal structure of a computer in which various embodiments of the disclosure may be implemented.

### DETAILED DESCRIPTION

As an overview, embodiments of a cannula system are comprised of three main parts: an outer cannula, an inner cannula, and an obturator. It should be noted that some embodiments may have a single cannula rather than separate inner and outer cannulas. The cannula or outer cannula is also known as the trocar or working channel and is essentially a tube that allows access to a surgical site.

In exemplary embodiments, the inner and outer cannula are made of silicone, plastic, or any other polymer that is safe for surgical procedures. Cannulas may be of combined solid (plastic or similar) and soft (e.g., silicone) materials. The obturator can be made of either plastic or metal, fully metal or just the tip made of metal. The cannula can come in different sizes tailored to specific procedures. For arthroscopy, an exemplary outer cannula has a width ranging from about 4 mm to about 15 mm and a length between about 30 mm and about 100 mm. For laparoscopy, an exemplary outer cannula has a width between about 3 mm and about 18 mm, and a length from about 40 mm to about 120 mm. Exemplary inner cannula are about 3-18 mm in diameter for orthopedics and about 3-15 mm in diameter for abdominal procedures. It should be noted that these are exemplary dimensions and the cannula could be larger or smaller depending on the procedure and may vary depending on the size of the joint, whether it be the knee, shoulder, hip, wrist, etc. When the surgeon uses a single portal, the cannula could be made significantly larger as needed.

Exemplary cannulas and obturators may be equipped with handles and/or locking mechanisms for case of use. Another commonality across all disclosed embodiments is that the cannula systems include one or more cameras, which, in each embodiment, may be static/fixed or deployable during insertion. The cameras could be tiltable to mitigate or eliminate potential dead zones between camera views. LEDs or other types of light sources may be provided in any of the disclosed embodiments, typically close to and aligned with the cameras. All embodiments could also have an irrigation connector for irrigating the surgical site. A processor or computer is provided to receive images from the cameras and provide the images in three dimensions. Advantageously, disclosed embodiments of the cannula system may come fully assembled in a sterile package so the surgeon does not need to assemble the system.

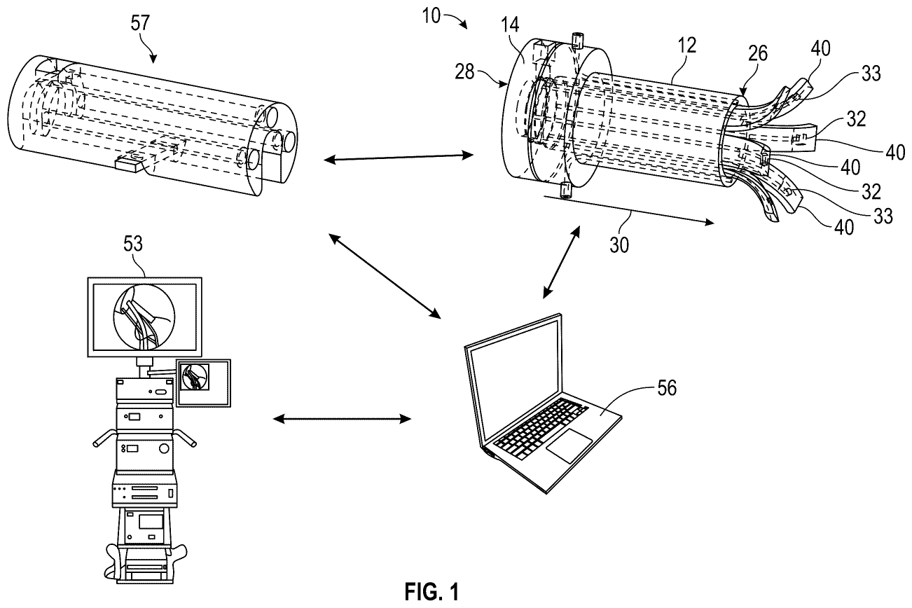

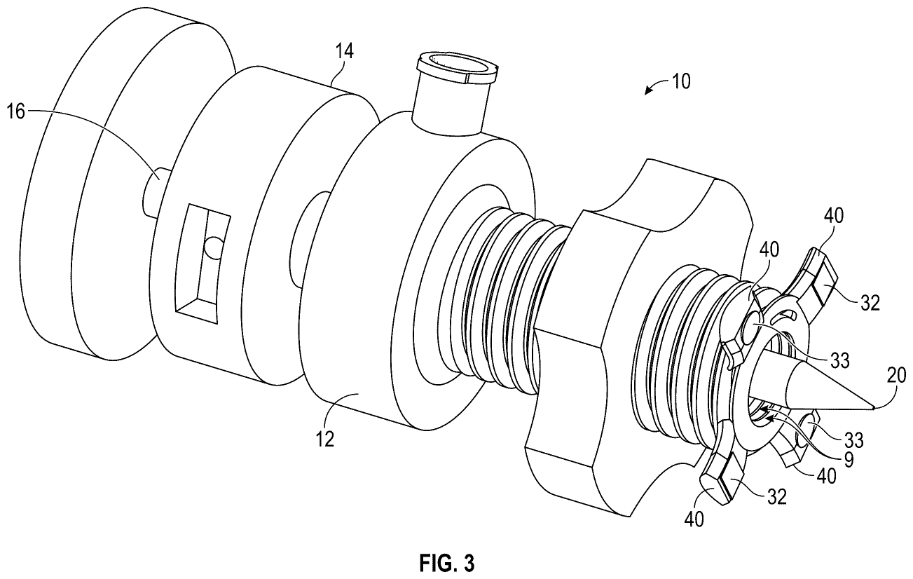

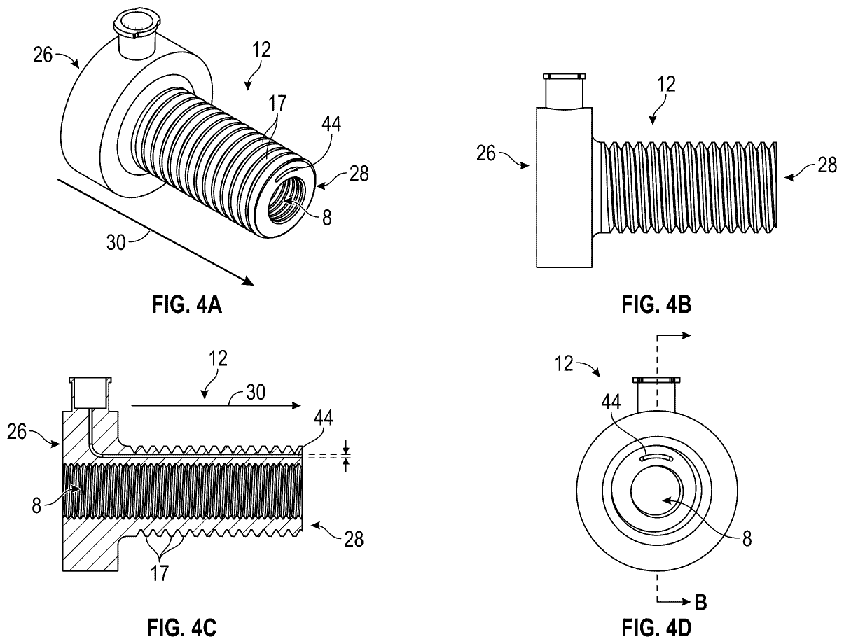

Referring to FIGS. 1-9F, exemplary embodiments of a cannula system 10 will now be described. The outer cannula 12 has a proximal end 26 relatively closer to the surgeon during use and a distal end 28 relatively closer to the patient during use. The insertion axis 30 of the outer cannula 12 is the axis representing a straight line between the surgeon and the patient. Outer cannula 12 has a central lumen 8 configured for insertion of an inner cannula 14. As described in more detail herein, when the cameras 32 are aligned with the insertion axis 30 they are essentially facing the surgical site from the perspective of the surgeon. Inner cannula 14 also has a proximal end 34 relatively closer to the surgeon during use, a distal end 36 relatively closer to the patient during use, and may define a central lumen 9. The inner cannula's insertion axis 38 also represents a straight line between the surgeon and patient.

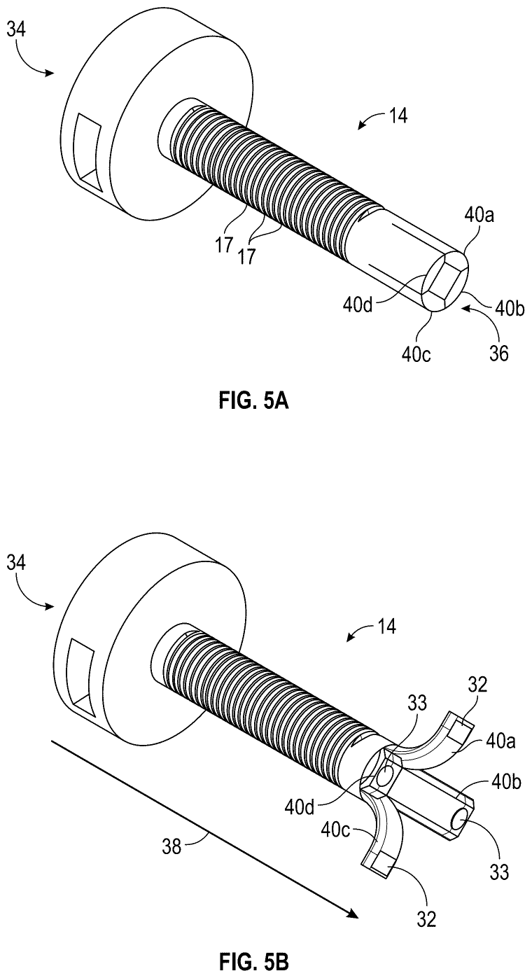

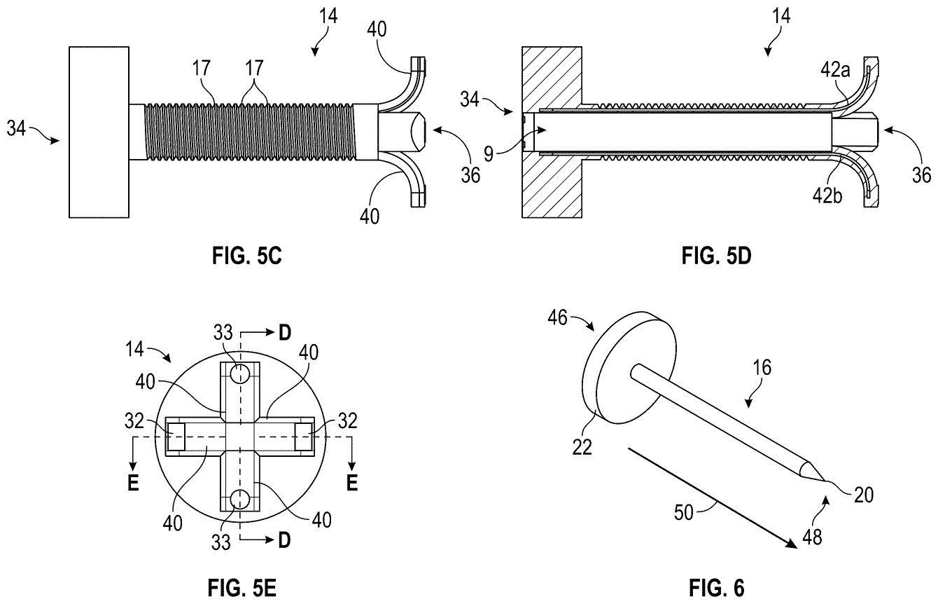

As best seen in FIG. 5D, the inner cannula 14 may have double lumens 42a, 42b, or tiny tunnels for wires. Each lumen 42a, 42b is separated and well-contained. Also, at the distal end 28 of the outer cannula 12, there may be one or more openings 44 for the passage of gas, water, and suction into the operation site. The inner cannula 14 is designed to fit inside the outer cannula 12 and, as described in more detail herein, is equipped to accommodate the camera module 32, one or more LEDs 33 or other lighting modules, and a battery at its proximal end. Wires from the camera module 32 are connected to the camera, sensors, and lights 33 at the distal end of the cannula. All wires are isolated and separated to ensure patient safety.

In exemplary embodiments, outer cannula 12 is a threaded component with threads 17 around its circumference to allow easier progress forward through different layers of tissue and to stabilize the cannula 12 once it is in place. Inner cannula 14 may also have threads 17 to stabilize it within the outer cannula 12. The inner and/or outer cannula may also have separate lumens to pass electricity, with appropriate safety measures in place. Valves may be integrated into the proximal part of the inner cannula 14 to prevent gas or water leakage from the operation site. These valves also provide easy access for surgical instruments or implants through the working channel. In the middle part of the inner cannula 14, a stabilizer can be included as part of the gimbal motor for the camera 32, and the camera could be tiltable to eliminate dead zones. This allows for 3D visualization and stabilization during the procedure. There may be at least one irrigation/suction connector, and inner or outer cannula 12, 14 defines one or more lumens 42a, 42b configured for insertion of irrigation connectors and passage of fluids or suction.

The obturator 16 typically is a rigid component that fits inside the central lumen 9 of the inner cannula 14 and has a potential cutting edge 20 to allow easy access through different layers of the operation site. Obturator 16 has a proximal end 46, a distal end 48, and an insertion axis 50 and is sized to be able to fit within the inner cannula 14. Typically, obturator 16 has a cutting edge 20 in the form of a sharp, cone-shaped tip for piercing the tissue of the patient. The obturator 16 may be equipped with an ergonomic handle 22 and a locking mechanism at the proximal end of the outer cannula 12.

In exemplary methods, after inserting the outer cannula 12, the obturator 16 is removed, and the inner cannula 14 is deployed in its place. This technique remains consistent in multiple embodiments, with the obturator 16 serving to protect the cameras 32 and keep them covered during deployment into the joint. Alternatively, outer cannula 12, inner cannula 14, and obturator 16 are inserted together and then the obturator is removed. Once the cannula system 10 is positioned deeply, the obturator 16 is withdrawn, allowing the cameras 32 to be activated inside the joint through a predefined positioning mechanism that expands them within the cavity.

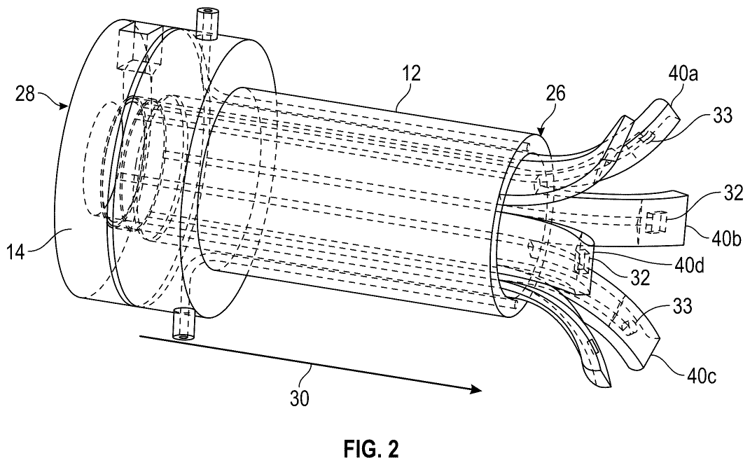

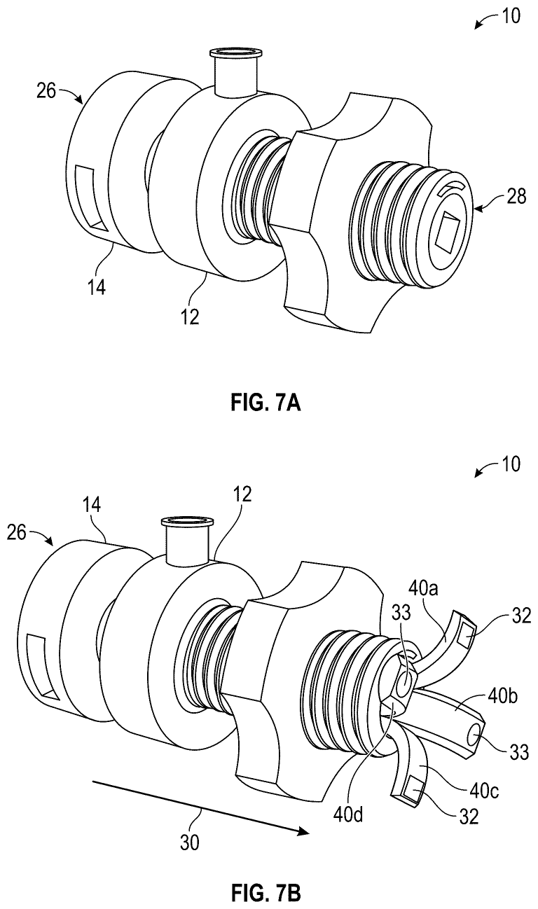

As best seen in FIGS. 2, 3, 5A and 5B, in this cannula system 10, cameras 32 and LEDs 33 are housed in the ends of positioning members (or arms) 40, which are part of the inner cannula 14. More particularly, the positioning members 40 are integrated with the distal end 36 of the inner cannula 14. In exemplary embodiments, there are four positioning members 40a, 40b, 40c, 40d movable between a closed position (see FIGS. 5A and 7A) and an open position (see FIGS. 5B and 7B), two cameras 32, and two LEDs 33. More LEDs could be provided as needed to allow the surgeon to see areas of interest. In the closed position, positioning members 40 are flush with the cylindrical surface of the inner cannula 14; in the open position they fold open to expose cameras 32 and align them with the insertion axis 30, i.e., face the cameras toward the surgical site.

In exemplary embodiments, positioning members 40 house not only cameras 32, but also sensors, one or more LED lights 33, and/or tiny motors for stabilization, as well as for tracking the movements of instruments in 3D. The positioning members 40 are movable and can be opened and closed according to the surgeon's preference. Once the cameras 32 are inserted, they automatically activate and transmit video images to the system. Exemplary embodiments may provide an option for the surgeon to manually activate and turn off the cameras 32. The cameras 32 could be tiltable to mitigate or eliminate potential dead zones in between the camera views.

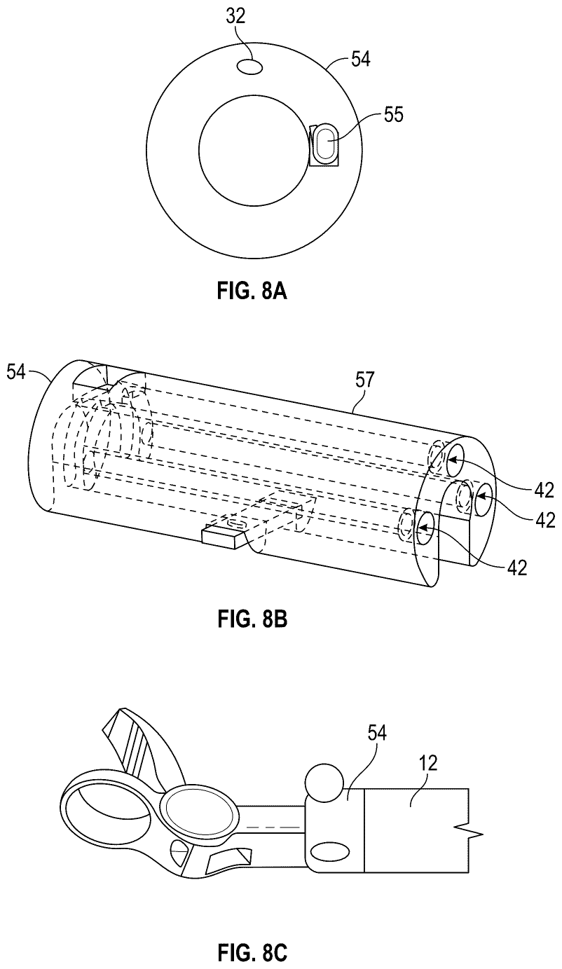

Disclosed cannula systems can be connected to a surgical instrument, as described herein. This advantageously allows the surgeon to have a closer view of the operation site and a close view of the movement of the surgical instrument. There are at least two options for making these connections. In exemplary embodiments, near the edge of the cannula 12, instead of arms there could be a ring 54 with a camera 32 housed in it ready to be hooked when an instrument passes through it. As shown in FIGS. 8A, 8B, and 8C, ring 54 is typically located at or near the distal end of the cannula 12. The ring 54 is hooked by the instrument, allowing it to be securely positioned, and may have a locking system 55 to aid with secure connection to the instrument. Once the surgeon removes the instrument, he or she can detach the ring 54 and prepare it for the next instrument. It is at the surgeon's discretion whether to use the ring 54 or not.

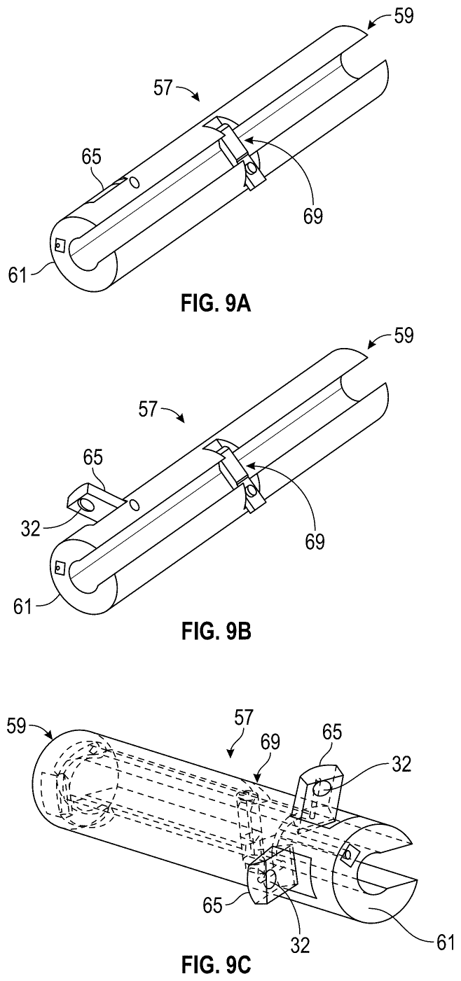

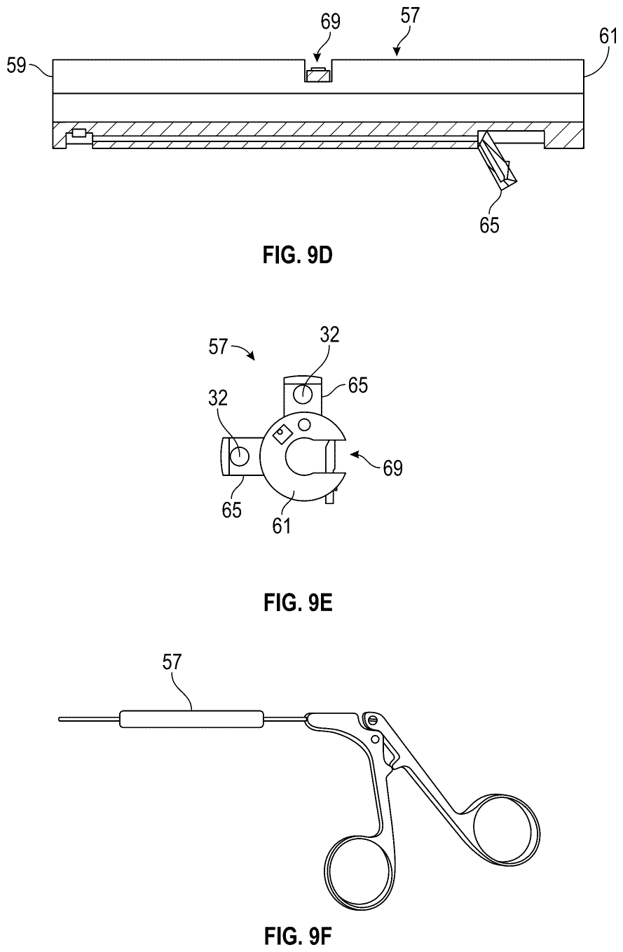

With reference to FIGS. 9A-9F, one option for deploying the cameras is a pre-connected camera instrument 57, which is separated from cannula system 10, and is a disposable part with quick connection to the instrument before it enters the body. This part is transferred between different instruments and transmits the image and video to the computer or processor 56 directly and then to a video screen 53. An exemplary camera instrument 57 has a proximal end 59, a distal end 61, an insertion axis 63, and one or more inner lumens 42. Camera instrument 57 may have a locking mechanism 69 to allow confirmation of the attachment to the device. A camera 32 is housed in or integrated with one or more small flaps 65, which may be located at or near the distal end 61 of the pre-connected camera instrument 57. The camera may be tiltable to mitigate or eliminate dead zones.

Flap 65 is movable between a closed position in which the camera 32 is protected and an open position in which it is exposed and aligned with the insertion axis 63. In the closed position, flap 65 is disposed in a slot 67 defined in the surface of the camera instrument 57, and in the open position the flap 65 lifts outward so it is angled relative to the instrument surface. There may be one flap 65 and camera 32 or multiple, on the top and one or more sides of the camera instrument 57. The camera 32 on this element has separate energy power from the cannula and is independent and supports the light source. This power is inside the casting and has one or more tiny tunnels. Exemplary embodiments include that the camera 32 is opened by electric order from the surgeon and closed when it can be in front of the system and is protected by the casing.

In exemplary embodiments, a processor 56 or computer is based near the operating bed and is the station for all data to come in from all cameras 32 to do a fast process to allow constant flow. This part of the system combines a direct transmission to the endoscopic tower and return to the cameras 32 about the movements of an instrument, so it provides the best pictures inside the body. In exemplary embodiments, the processor 56 has an AI feature to provide the best images to the surgeon, to allow 3d images to constantly be provided, and surgical feedback/suggestions related to the operation type. For example, choosing the right place to drill a hole, or to notice the beginning of bleeding, and more.

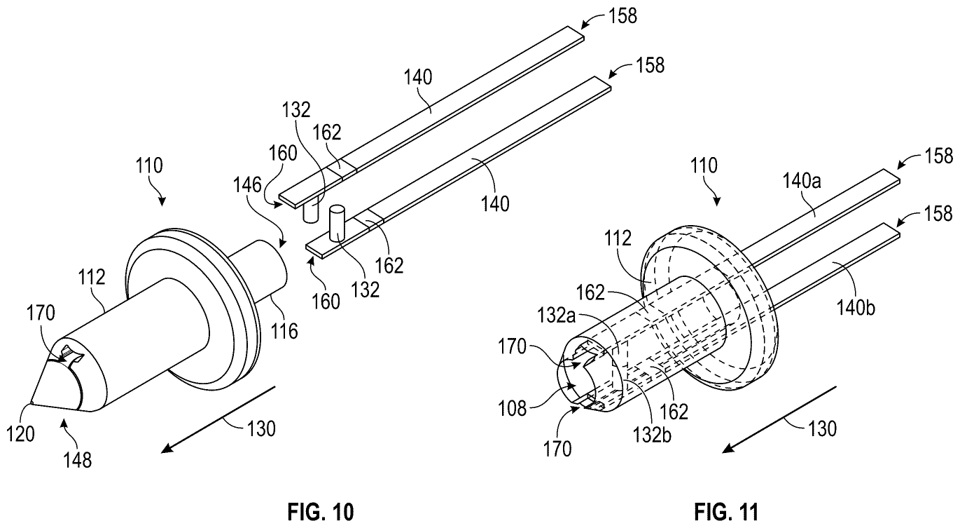

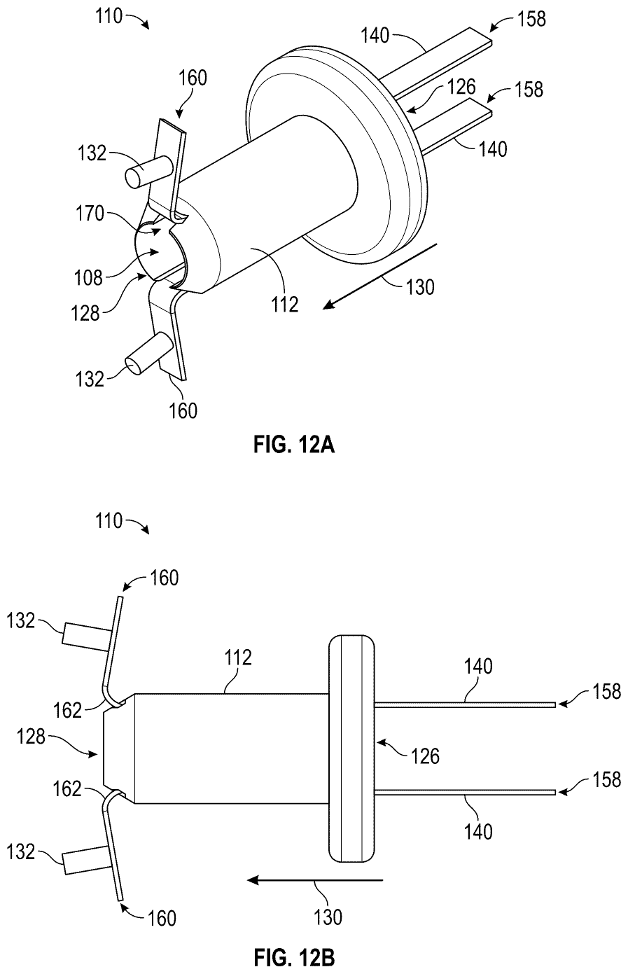

Turning to FIGS. 10-13B, exemplary embodiments deploy cameras 132 using movable positioning members 140 configured to run through the cannula 112. In these systems, each camera/chip 132 may be mounted on a positioner/introducer 140, which may be wired to a processing module located at the back of the cannula/trocars 112 or may connect directly to an external box positioned outside the operating field that houses a video processor. In exemplary embodiments, one or more LEDs (Light Emitting Diodes) may be positioned next to the cameras to illuminate the concealed surgical field.

In exemplary embodiments, cameras 132 are placed on pre-shaped positioning members 140 made of Nitinol, plastic, silicon, or other materials. The positioning member 140 may be a strip positioner or other shapes, like a half circle or U-shape that holds the cameras 132. Typically, the cannula system 110 has two positioning members 140a, 140b, but one might suffice in some applications and up to eight could be used. Each positioning member 140 has a proximal end 158, a distal end 160, and a flexible portion 162 near the distal end 160. The flexible portion 162 allows for bending or outward folding of the distal end 160 of the positioning member 140 as described herein. A camera 132 is connected to each positioning member 140a, 140b at or near its distal end 160. In certain embodiments, the cameras 132 and their positioning members 140 are pre-connected and hidden at this stage in cannula internal grooves/slots 170.

The cannula 112 has a proximal end 126 relatively closer to the surgeon during use and a distal end 128 relatively closer to the patient during use. The insertion axis 130 of the cannula 112 is the axis representing a straight line between the surgeon and the patient. Cannula 112 has a central lumen 108 configured for insertion of the obturator 116. In exemplary embodiments, slots 170 are defined in the distal end 128 of the cannula 112. These are sized to receive positioning members 140 when they are in their deployed position. More particularly, the flexible portion 162 of each positioning member 140 mates with or sits in the respective slot 170 when the distal end 160 of the positioning member 140 folds outward to deploy the camera 132. Cannula 112 may also have a circular flange or handle 122 at its proximal end for case of use.

Additionally, at the back of the cannula 112, there may be at least one irrigation/suction connector, and fluids or suction pass through the central lumen 108 of cannula 112. Exemplary embodiments have a processing module and a battery to enable wireless operation or a connector to connect a cable for power supply and video streaming to an external computing box located near the operating field. The computing box may be connected to a large monitor.

As best seen in FIG. 11, when positioning members 140 and cameras 132 are in their undeployed position, the cameras 132 are positioned perpendicular to the insertion axis 130 such that the face or lens of the cameras 132 face each other. The camera 132 faces or lenses may be flush with each other when undeployed inside the outer cannula 112. In the deployed position, the flexible portion 162 of the positioning member 140 is bent and folded outward. In this position, the cameras 132 are aligned with the insertion axis 130, i.e., the camera 132 faces, or its lenses become aligned with, insertion axis 130 and face the surgical site. The cameras 132 could be tiltable to mitigate or eliminate potential dead zones in between the camera views.

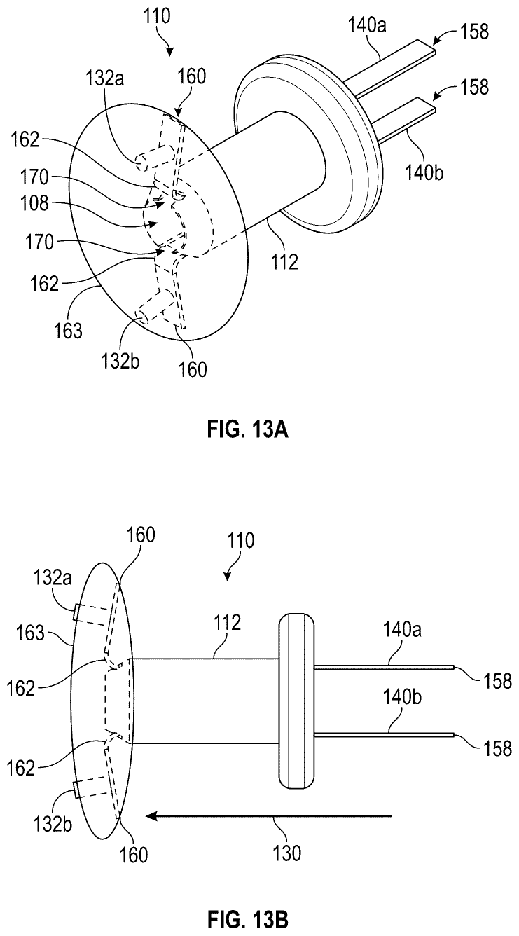

The positioning members 140 can exert forces to either side of the cannula 112 or glide laterally and lock mechanically to the cannula or any other supporting system. The expended positioning members 140 are used both to position the two cameras 132 in parallel positions and to hold the surrounding tissue from “falling” into the field of view and obstructing the video image. As shown in FIGS. 13A and 13B, a flexible disc 163 may be provided to protect the positioning members 140.

An obturator 116 is provided and has a proximal end 146, a distal end 148, and an insertion axis. It can be made of either plastic or metal, fully metal or just the cone-shaped tip made of metal. The obturator 116 is a generally conical component with a cone-shaped distal end 148 having a cutting edge 120 to penetrate body tissue of the patient. In some embodiments, the obturator 116 may have slots or holes to fit the cameras and allow them to face the surgical site. In exemplary embodiments, the cannula opens at the joint by entering tunnels in a silicone ring. An additional approach involves the obturator featuring slots or holes for live camera insertion. This allows the camera to be introduced during the cannula's placement.

In operation, the procedure begins with insertion of the cannula 112 without the cameras 132. The surgeon or nurse inserts the cannula 112 into the surgical site and slides the obturator 116 into the central lumen 108 of cannula 112 so the cone-shaped distal end 148 of the obturator extends from the distal end 128 of the cannula 112. The insertion is facilitated by the cone-shaped obturator 116 and its cutting edge 120. After using the cutting edge 120 of the obturator 116 to cut tissue of the patient as needed, the surgeon withdraws the obturator 116 from the cannula 112 by sliding it in a proximal direction and out the proximal end 126 of the cannula.

Once the cannula 112 is securely positioned and the obturator 116 withdrawn, the camera/chip 132 can be connected to the system. Thus, the surgeon next inserts positioning members 140a, 140b into the cannula 112. The surgeon may hold positioning members 140 and cameras 132 in their undeployed position in which the cameras 132 are positioned perpendicular to the insertion axis 130 such that the faces or lenses of the cameras 132 face each other. The surgeon may then slide the positioning members 140a, 140b further in the cannula 112 along its insertion axis 130 in the distal direction to deploy the cameras 132a, 132b.

When the distal ends 160 of the positioning members 140 exit the distal end 128 of the outer cannula 112 to deploy the cameras 132, the flexible portion 162 bends and folds outward. This causes the cameras to move from positions perpendicular to the insertion axis 130 to positions aligned with the insertion axis 130, that is the camera 132 faces or lenses become aligned with insertion axis 130 and face the surgical site.

Thus, cameras 132 can be deployed and inserted within the cannula 112, allowing them to be activated within the joint or cavity space. Once positioning members 140 are fully positioned in place, a parallel view from the two cameras 132a, 132b is displayed on the monitor. As best seen in FIGS. 13A and 13B, the deployed cameras 132a, 132b advantageously provide a large radius for viewing by the surgeon. After completing the procedure, the surgeon or nurse retracts positioning members 140a, 140b and removes the cannula 112 from the surgical site.

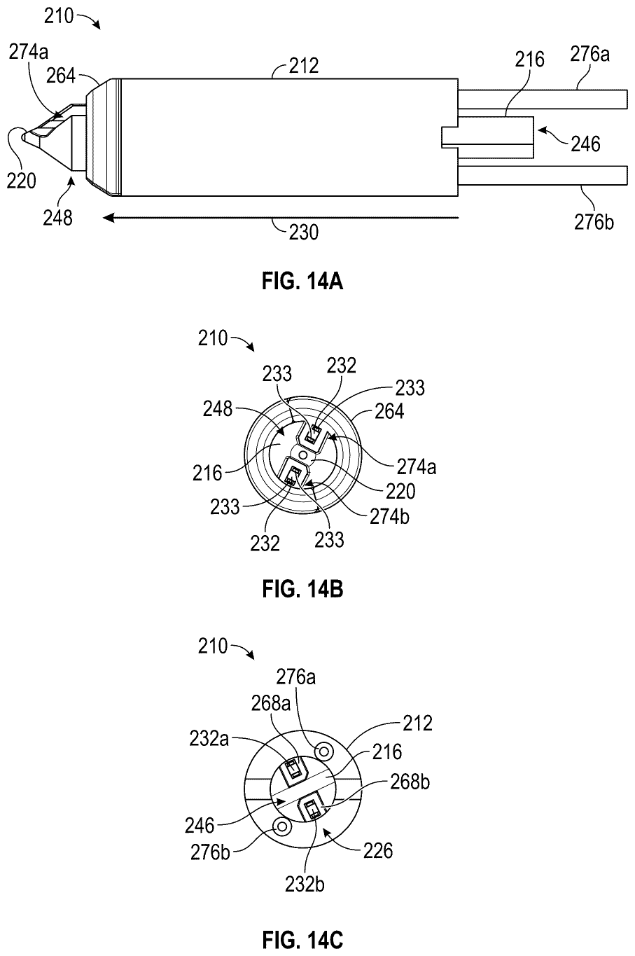

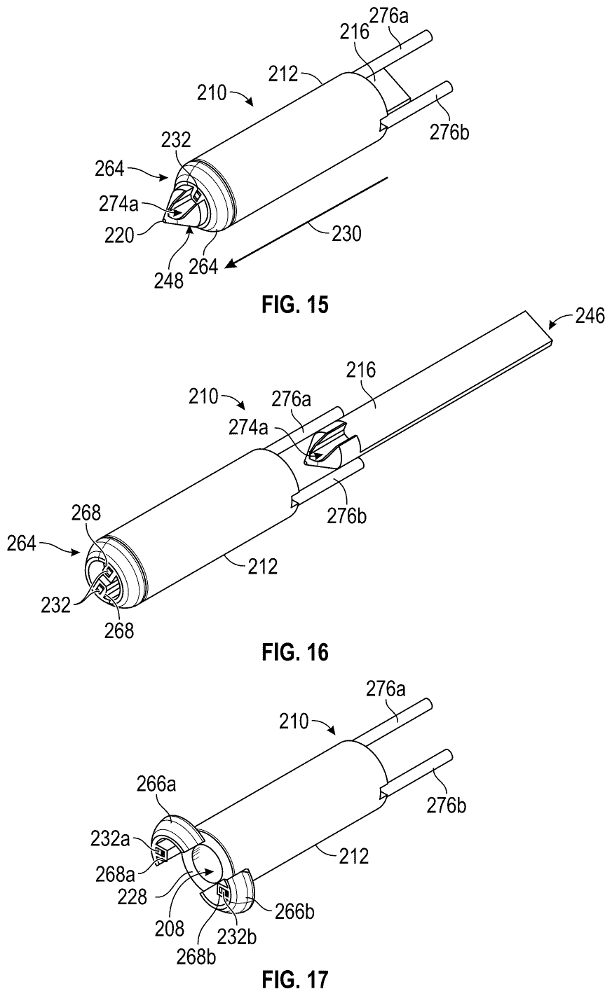

With reference to FIGS. 14-18B, exemplary embodiments of a cannula system 210 advantageously provide under vision insertion by housing cameras 232 in a movable disc 264 so the cameras are always facing the surgical site. More particularly, the distal end 228 of the cannula 212 (the part that is in the body) is a movable disc 264. Disc 264 can be integrally formed with the cannula 212 or could be a separate component pre-connected to the cannula 212 at its distal end 228.

Cannula 212 has a proximal end 226, a distal end 228, and an insertion axis 230 representing a straight line between the surgeon and the patient. The cannula 212 also has a central lumen 208 configured for insertion of the obturator 216. The face of disc 264 is aligned with the insertion axis 230. The cannula system 210 also includes an obturator 216 and one or more rotating handles 276, which are used to separate half portions of the disc 264, as described in more detail herein.

An exemplary disc 264 is comprised of two separable half portions 266a, 266b, which are movable between a first position together flush with each other and a second position in which they are separated from each other. Disc 264 is essentially O-shaped with a space in the center and flanges 268 extending into the center of the O. Exemplary embodiments have two flanges, and each flange 268a, 268b defines a slot 270 therein to house a camera 232 and one or more LED lights 233. Several LEDs 233 could be provided as needed to allow the surgeon to see areas of interest.

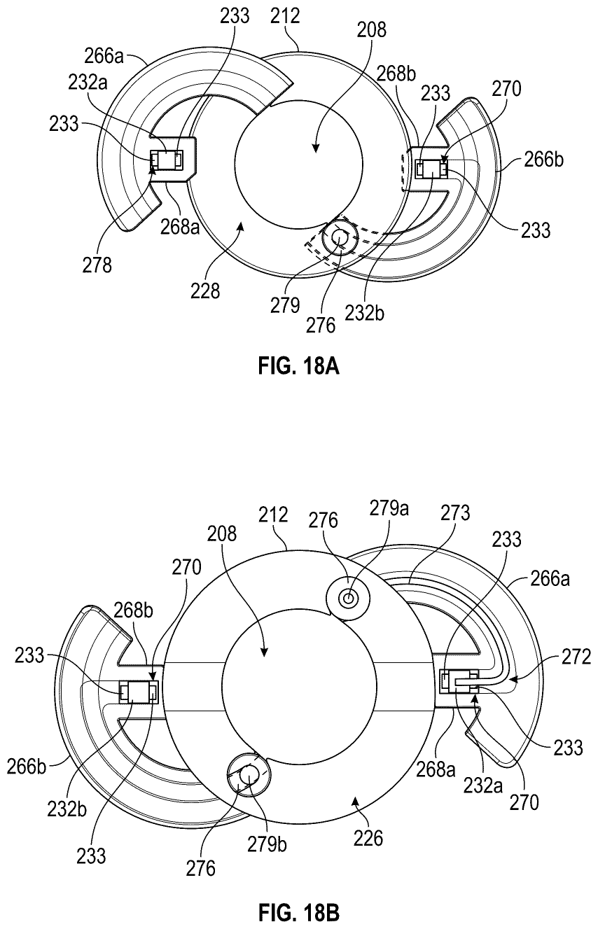

Thus, each separable half portion 266a, 226b houses a respective camera 232a, 232b and LED lights 233 and, because disc 264 is aligned with the insertion axis 230 of the cannula 212, the cameras 232 are also thus aligned and face the surgical site. Cameras 232a, 232b could also be tiltable to mitigate or eliminate dead zones. As best seen in FIG. 18B, disc 264 has a semi-circular channel 272 or hollow portion within it. This channel 272 can be used to house wires 273 or circuitry as needed. An irrigation connector may be provided for flow of air, water, or suction as needed. Each half portion 266a, 266b may have a rotating connector 279 connecting it to the distal end 228 of the cannula 212.

Obturator 216 has a proximal end 246 and a distal end 248 and is configured to be disposed within the central lumen 208 of cannula 212. It could be substantially conical or substantially flat with a cone-shaped distal end 248. The cone-shaped distal end 248 has a cutting edge 220 to penetrate body tissue of the patient. In exemplary embodiments, there are channels 274a, 274b formed in the cone-shaped distal end 248 of the obturator 216. These are sized and located to be aligned with the cameras 232 so each camera has an unobstructed view of the surgical site along the insertion axis 230. The obturator 216 also serves to protect the cameras 232 and keep them covered during deployment into the surgical site.

In operation, the surgeon inserts obturator 216 into the central lumen 208 of cannula 212 so each channel 274a, 274b is aligned with a respective camera 232a, 232b housed in each slot 270 in a respective flange 268a, 268b of each half portion of the movable disc 264. Then cannula system 210 is inserted by the surgeon into the surgical site. As mentioned above, cameras 232 are facing the surgical site and are live during insertion of the cannula system 210, providing under vision insertion capability. Thus, a major advantage of disc embodiments is that the cameras 232 are “looking” forward during the cannula insertion phase, allowing the surgeon full visual coverage of the entire procedure from the insertion phase, rather than just starting after the full positioning of the cannula. The video starts as soon as the insertion starts (or before), allowing the surgeon to stop at any stage and validate the final position of the cannula. The surgeon has a live feed from the cameras 232 from the moment he or she starts insertion of the cannula system 210, if not before.

If needed, the surgeon can cut tissue of the patient using the cutting edge 220 of obturator 216. The surgeon can continue to push the cannula system 210 deeper into the surgical site as needed. Once the cannula system 210 is positioned deeply and the surgeon is finished with the obturator 216, he or she withdraws it from the central lumen 208 of cannula system 210 by pulling it out of the cannula 212 via its distal end 228. When the cannula 212 is fully inserted and the obturator 216 is removed, the surgeon can slide apart the two separable half portions 266a, 266b of the disc 264 by rotating handles 276a, 276b located on the proximal end of the cannula, which in turn rotates rotation axes of the rotating connectors 279a, 279b. This advantageously expands the view of the cameras 232 within the cavity/surgical site, providing the surgeon with a larger field of view. The separation and expansion of half portions 266a, 266b also serve to hold the surrounding tissue from “falling” into the field of view and obstructing the video image.

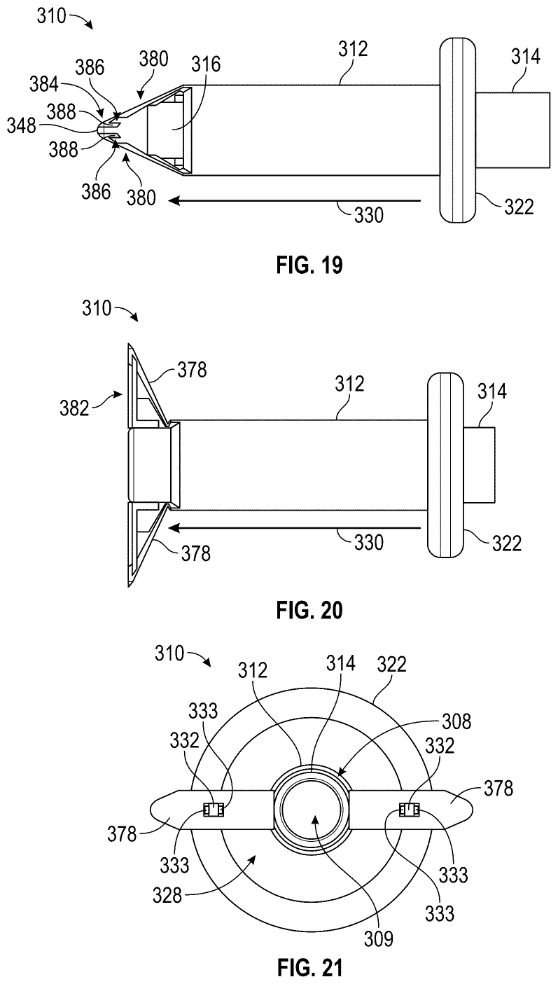

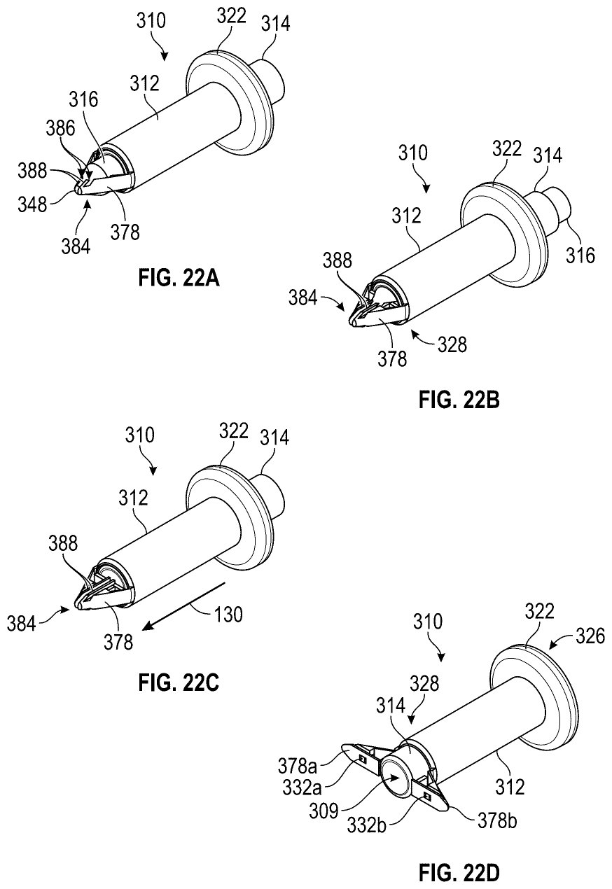

Turning now to FIGS. 19-22D, cannula system 310 houses cameras 332 in movable flaps 378 located at the distal end 328 of a single cannula 112 or outer cannula 112. This system 310 is comprised of a single cannula or outer cannula 112 having a proximal end 326, a distal end 328, a central lumen, 308, an insertion axis 330, and flaps 378 attached to or integrally formed with the distal end 328. A single flap or several-up to six or even eight flaps-could be used, and exemplary embodiments employ two flaps 378a, 378b. Each flap 378a, 378b houses a camera 332 and could house one or more LED lights 333. Cameras 332 may be tiltable to eliminate potential dead zones between each camera view. The number of LEDs 333 could vary and would be enough to allow the surgeon to see all areas of interest. In exemplary embodiments, flap 378 is essentially the shape of a right triangle with a camera housed at about the middle of the long edge.

In exemplary embodiments, an inner cannula 314 and obturator 316 also are provided. The inner cannula 314 is configured to be disposed within the central lumen 308 of the outer cannula 312 along the insertion axis 130, and the obturator 316 is sized to fit within the central lumen 309 of inner cannula 314. The outer cannula 312 may also have a circular flange or handle 322 at its proximal end for case of use. As with other embodiments, the outer cannula may have inner lumens for inserting an irrigation connector and a processor in communication with the cameras. It should be noted that this embodiment could be provided with a single cannula.

As best seen in FIGS. 19-20 and 22C-22D, flaps 378 can be moved between a closed position 380 and an open position 382 by virtue of joints, spring action, or other suitable mechanisms. When the obturator 316 is disposed within the central lumen 309 of inner cannula 314, it holds flaps 378 in the closed position 380 by a locking or mating mechanism 384 comprised of mating slots 386 in the distal end or tip 348 of the obturator 316 and corresponding mating members 388 in the flaps 378. In the closed position 380, i.e., when obturator 316 is fully inserted in the central lumen 309 of inner cannula 314, the flaps 378 and the two cameras 332a, 332b housed therein face the obturator and are flush with the tip 348 of the obturator 316. As best seen in FIG. 19, in the closed position 382, the hypotenuses of the two flaps 378 form a point together with the tip 348 of obturator 316.

As the obturator 316 is withdrawn, the mating mechanism 384 unlocks and flaps 378 move to the open position 382 where they, and cameras 332, are aligned with the insertion axis 330 and face the surgical site. More particularly, when obturator 316 is removed from the inner cannula 314, the long edges of the flaps 378 move closer to each other, initially into a parallel position. Then, pushing the inner cannula 314 forward so its distal end pushes the flaps 378 moves the flaps to their open position 382, thus exposing the cameras 332 so they face the surgical site. In this position, the inner cannula 314 maintains the flaps 378 in their open position.

In operation, the surgeon or nurse inserts inner cannula 314 into the central lumen 308 of outer cannula 312 and obturator 316 into the central lumen 309 of inner cannula 314. The surgeon or nurse pushes obturator 316 through the outer cannula 312 so the obturator tip 348 extends beyond the distal end of the outer cannula and the mating mechanism 384 engages and holds flaps 378 in the closed position for insertion. More particularly, the mating mechanism 384 with its mating slots 386 in the tip 348 of the obturator 316 and corresponding mating members 388 in the flaps 378 holds the flaps 378 in the closed position 380. The surgeon or nurse stops pushing inner cannula 314 when the distal end of the inner cannula 314 is still disposed within the central lumen 308 of outer cannula 312 or flush with the distal end of the outer cannula 312 so as not to disengage the mating mechanism 384 and open the flaps 378 prematurely. The full cannula system 310 is then inserted into the surgical site.

Once the cannula system 310 is positioned deeply enough in the surgical site and the surgeon is finished with the obturator 316, he or she withdraws it from the cannula system 310 by pulling it out of the central lumen 309 of inner cannula 314 via its distal end 328. This unlocks the mating mechanism 384, i.e., mating members 388 in flaps 378 are separated from their corresponding mating slots 386 in the tip 348 of the obturator 316, and causes the long edges of the flaps 378 to move closer to each other into a parallel position where they, and the cameras 332, are facing each other. Then, after the obturator 316 is removed, the surgeon or nurse pushes the inner cannula 314 forward so its distal end pushes against the long edges of flaps 378, moving the flaps into their open position 382. In the open position 382, the long edges of the flaps 378 and the cameras 332a, 332b are aligned with the insertion axis 330 and face the surgical site.

Thus, cameras 332 are deployed, allowing them to be activated within the joint or cavity space. Once flaps 378 are opened, a parallel view from the two cameras 332a, 332b is displayed on the monitor. Flaps 378 are maintained in the open position 382 for the surgical procedure. After completing the procedure, the surgeon or nurse retracts the inner cannula 314, which causes the flaps 378 to move back from the open position 382 to the closed position 380 in which their long edges are parallel to each other and the cameras 332 are facing each other and protected. The surgeon or nurse then removes the remaining components of the cannula system 310 from the surgical site.

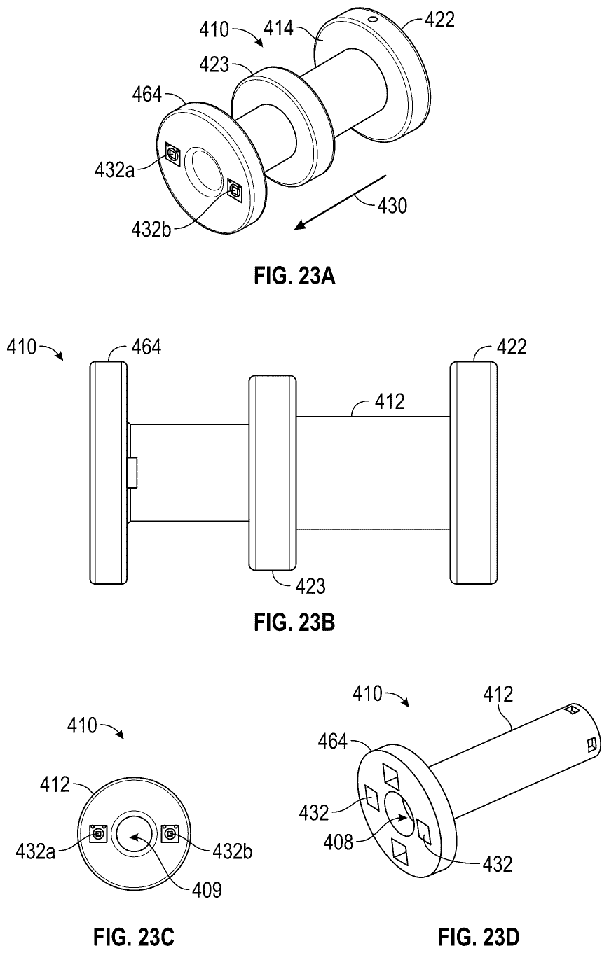

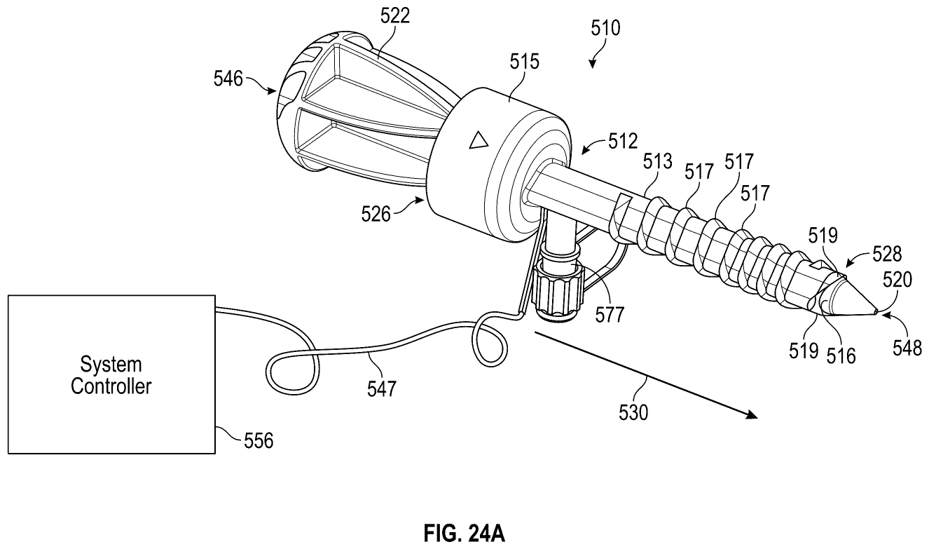

With reference to FIGS. 23A-23D, another exemplary cannula system 410 provides under vision insertion by virtue of cameras 432 aligned with the insertion axis 430 of the outer cannula 412. Outer cannula 412 has a proximal end 426, a distal end 428, a central lumen 408, and an insertion axis 430. The distal end 428 of outer cannula 412 forms a disc 464 for housing one or more cameras 432. In exemplary embodiments, cannula system 410 has two cameras 432a, 432b housed in disc 464. Advantageously, the cameras 432 are aligned with the insertion axis 430 of the outer cannula 412 facing the surgical site so the surgeon can view the surgical site during insertion of the cannula system 410.

The positioning of cameras 432a, 432b may vary depending on the application, and in exemplary embodiments, the cameras 432a, 432b are positioned at about 9 o'clock and about 3 o'clock of the disc 464. For certain applications, such as shoulder surgery, the cameras may be positioned at about 12 o'clock and about 6 o'clock of the disc. The distance between the two cameras 432a, 432b may be about 12-20 mm and in exemplary embodiments, is 16 mm. The distance could vary depending on the procedure and joint being operated on. The cameras 432a, 432 may be tiltable to mitigate or eliminate dead zones.

An inner cannula 414 is provided and configured to be disposed within the central axis 408 of outer cannula 412. Inner cannula 414 may have a central lumen 409 for receiving other surgical instruments and a circular or disc-shaped flange or handle 422 at its distal end for case of use. Another flange 423 that rings the center of the outer cannula 412 also may be provided to attach the cannula system 410 to the skin of the patient, thereby stabilizing it and keeping it in place. Exemplary dimensions of the outer cannula 412 are about 5-10 mm for its inner diameter, about 8-15 mm for its outer diameter, and about 20-30 mm for the flange diameter, though a range of dimensions are possible. In exemplary embodiments, the approximate length of the cannula system 410 is 30-60 mm for the outer cannula 412 and 20-50 mm for the inner cannula 414, but the dimensions may vary depending on the procedure and joint size.

In operation, the nurse or surgeon slides inner cannula 414 into the central lumen 408 of outer cannula 412 and then inserts the cannula system 410 into the surgical site. The cameras 432 are statically positioned along the insertion axis 430 facing the surgical site and are live during insertion of the cannula system 410, providing under vision insertion capability. Thus, cameras 432 are “looking” forward during the cannula insertion phase, allowing the surgeon full visual coverage of the entire procedure from the insertion phase, rather than just starting after the full positioning of the cannula.

The video starts as soon as or before insertion starts, allowing the surgeon to stop at any stage and validate the final position of the cannula. The surgeon has a live feed from the cameras 432 from the moment he or she starts insertion of the cannula system 410, if not before. The surgeon can continue to push the cannula system 410 deeper into the surgical site as needed. After completing the procedure, the surgeon or nurse retracts the cannula system 410 from the surgical site.

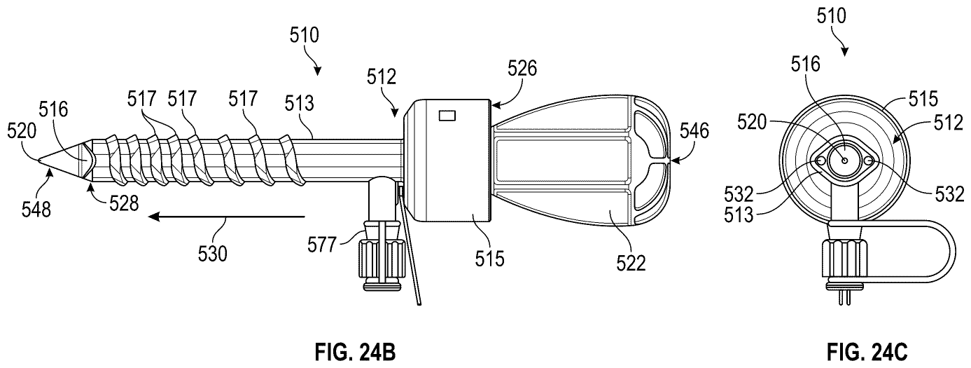

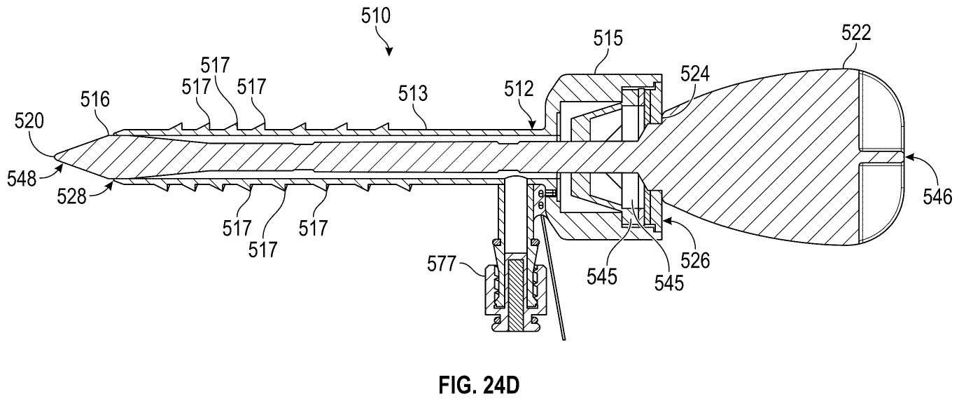

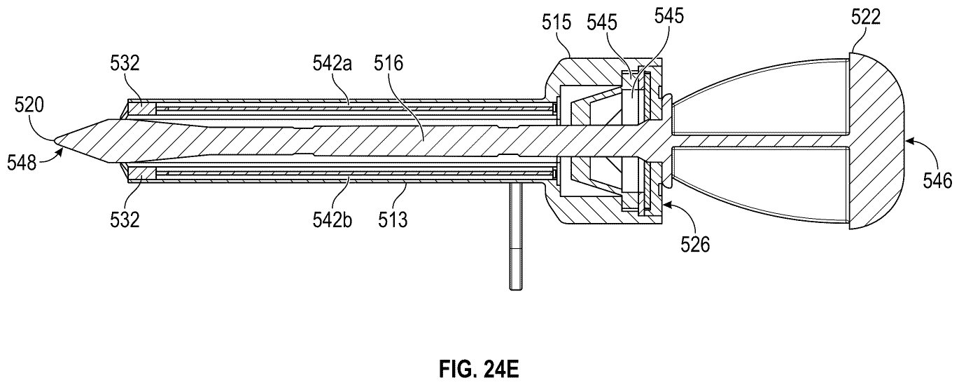

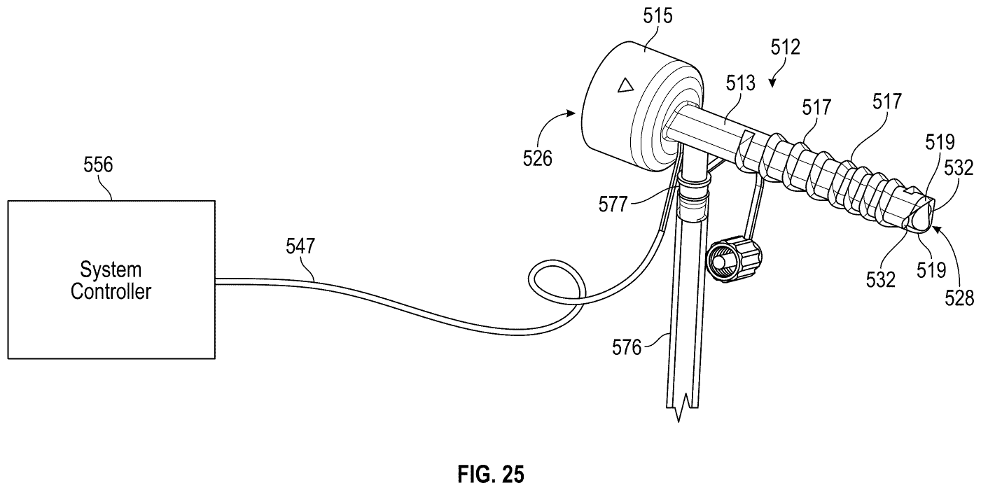

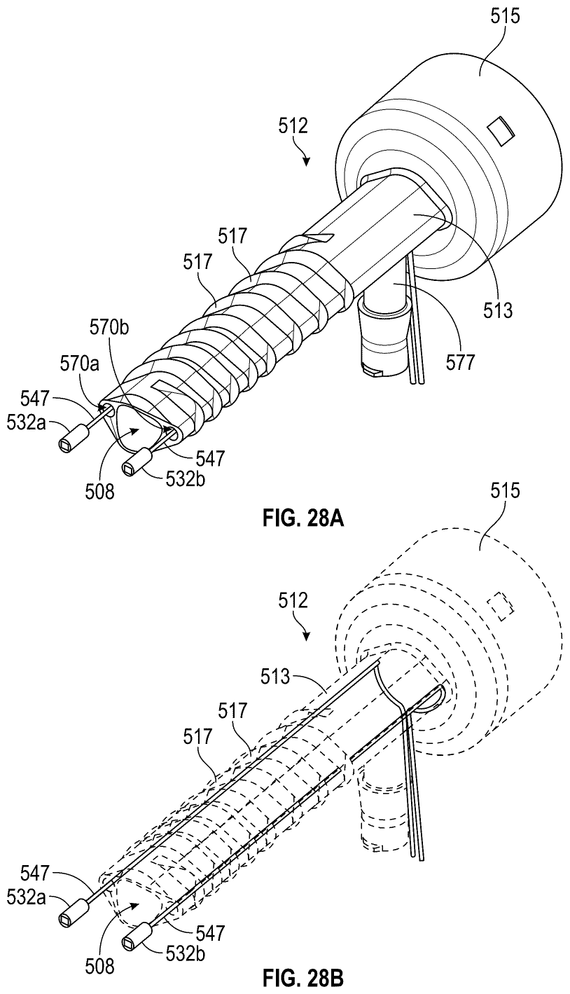

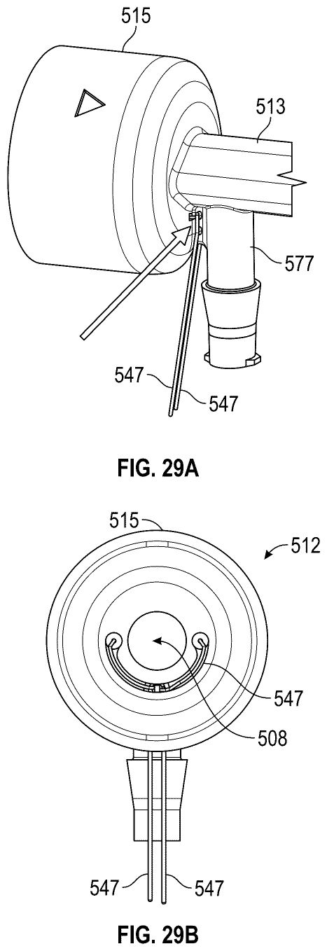

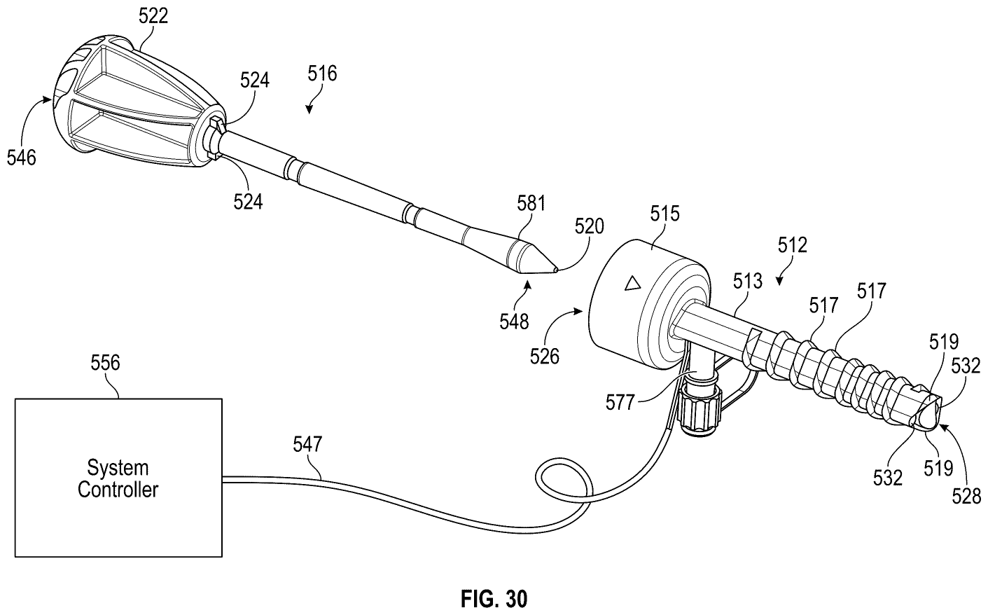

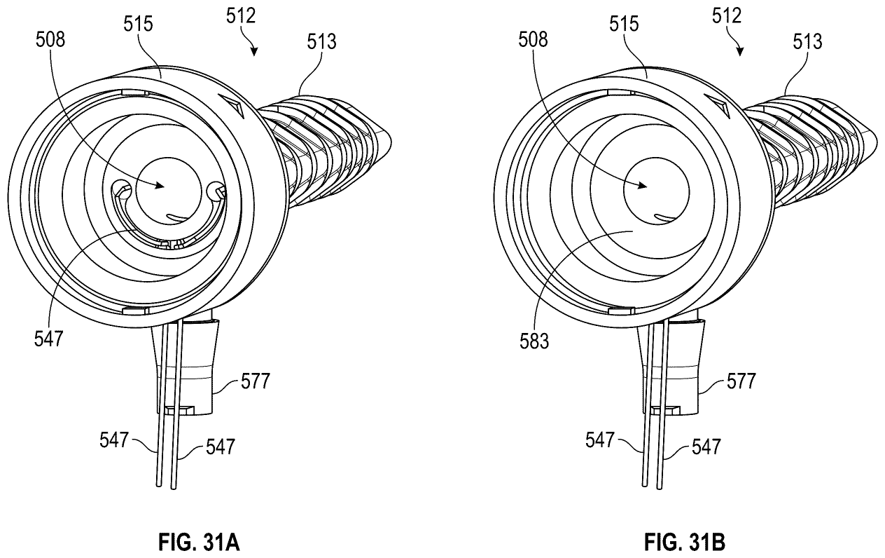

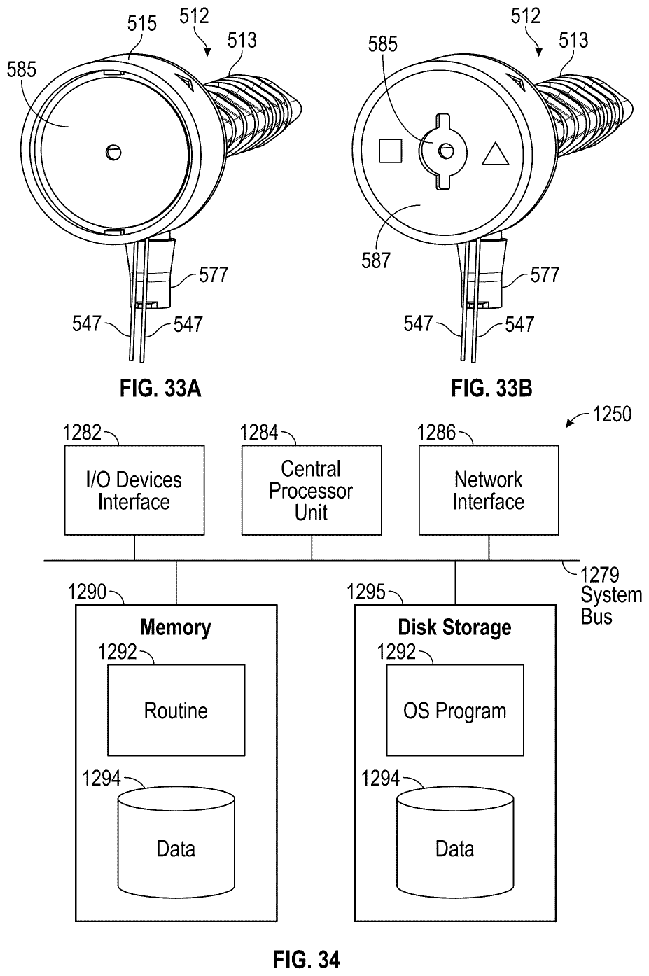

Turning to FIGS. 24A-33B, an exemplary cannula system providing static camera placement will now be described. The main component of cannula system 510 is cannula 512, also known as a trocar or working channel, that has a body portion 513 and a rear portion 515. Cannula 512 has a proximal end 526, a distal end 528, a central lumen 508, and an insertion axis 530. The cannula 512 typically is made of silicone, plastic, or other flexible or rigid polymers, or a combination of two or more polymers and/or of flexible and rigid polymers. In exemplary embodiments, the body portion 513 of the cannula 512 is a threaded component. More particularly, the middle part of the cannula body 513 has threads 517 around its circumference to allow easier progress forward through different layers of tissue and to stabilize the cannula 512 once it is in place.

Cannula 512 may be provided in different sizes, depending on the surgical procedures. For arthroscopy, cannula 512 has a width ranging from about 4 mm to about 15 mm and a length between about 30 mm and about 100 mm. For laparoscopy, an exemplary width is between about 3 mm and about 18 mm, and the length is between about 40 mm and about 120 mm. Additional exemplary dimensions are about 4-12 mm for the working channel and about 8-14 mm for the spacing of the two cameras 532. An exemplary rear part of the cannula 512 ranges in length from about 10 mm to about 45 mm, and an exemplary cannula body 513 ranges from about 50 mm to about 100 mm. In exemplary embodiments, vertical length is about 8-12 mm and horizontal length is about 10-20 mm. It should be noted that these are exemplary dimensions and the cannula could be larger or smaller depending on the procedure and may vary depending on the size of the joint, whether it be the knee, shoulder, hip, wrist, etc. When the surgeon uses a single portal, the cannula could be made significantly larger as needed.

An obturator 516 with a proximal end 546 and a distal end 548 is configured to be disposed within the central lumen 508 of cannula 512 along the insertion axis 530. Obturator 516 is a rigid component which may be equipped with a cutting edge 520 at its distal end 548 to allow easy access through different layers at the operation site. Its proximal end forms an ergonomic handle 522, and it may have a locking mechanism 524 that lines up with the proximal end of the cannula 512 when disposed within the cannula. Near the distal end 548 the obturator 516 may have a larger diameter or thicker portion 581 than the rest of the obturator body serving to better secure it when it is disposed within the cannula 512.

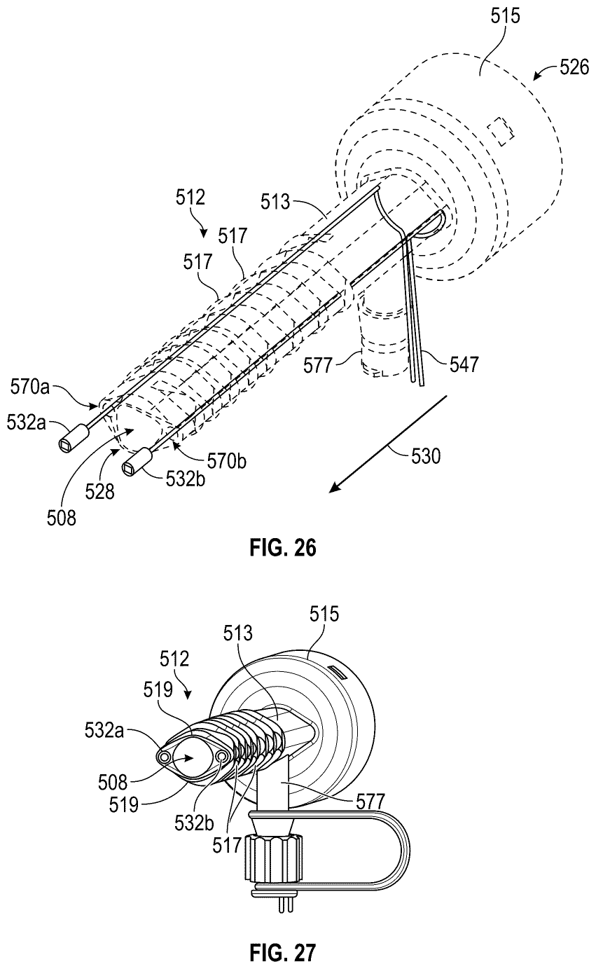

As best seen in FIGS. 26-28B, cameras 532 are housed in the distal end 528 of the cannula 512. The distal end 528 of cannula 512, which is an integral part of the cannula, stores the cameras 532 as well as one or more LED lights and tiny motors for stabilization. The cameras 532 could be tiltable to eliminate dead zones in the surgeon's vision. The number of LED lights will vary depending on how many are needed to illuminate all areas of interest. Exemplary embodiments employ two cameras 532a, 532b, which are housed in small slots 570a, 570b located in the distal end 528 of the cannula 512. Advantageously, the cameras 532 are statically positioned so they do not require separate deployment steps to be aligned with the insertion axis 530. Optionally, a gimbal motor for the camera 532 may be provided to improve 3D visualization and image stabilization during the surgical procedure.

The cannula body 513 is advantageously sized and shaped to house and best position two cameras 532a, 532b and allow space for the obturator 516 in the central lumen 508 while also maintaining a compact size. More particularly, cannula body 513 has a substantially diamond-shaped cross section, which provides a large enough central lumen 508 in the center of the diamond for the obturator 516 to be disposed within the cannula body 513 and leaves ample space in the opposite corners of the diamond for the slots 570a, 570b to house cameras 532a, 532b. Top and bottom overhangs 519 at the distal end 528 of the cannula body 513 serve to hold the surrounding tissue from “falling” into the field of view and obstructing the video image.

As best seen in FIG. 24E, in exemplary embodiments, cannula 512 has double lumens 542a, 542b consisting of tiny tunnels that allow for the passage of wires for electrical power and data transfer. Each lumen 542a, 542b is separated and well-contained. If needed, gas, water, and/or suction may pass through central lumen 508 of cannula 512 into the surgical site. One or more irrigation tubes 576 can be connected to the cannula system 510 by inserting the tubes into a port 577 or irrigation connector extending from the bottom of the body 513 of the cannula 512.

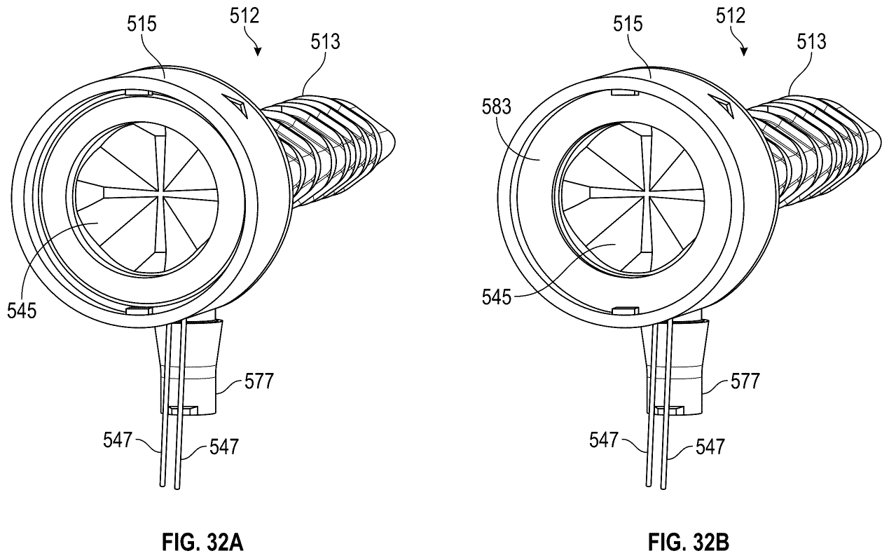

Rear portion 515 of cannula 512 is hollow and configured to house various components. As best seen in FIGS. 32A and 32B, one or more valves 545 may be integrated into the rear portion 515 of the cannula 512, preferably in the proximal part of the rear portion. The valves 545 prevent gas and/or water leakage from the surgical site and provide easy access for surgical instruments or implants through the cannula 512. Valves 545 may be cross-slit valves, and a seal 585 and seal cover 587 may also be provided.

The rear portion 515 of the cannula 512 also is equipped to accommodate wires 547 for conducting electricity and for the video stream as well as a wire cover 583 if needed. Electronic module wires 547 are connected to the cameras 532 as well as to any sensors and lights that can be located at or near the distal end 528 of the cannula 512. Advantageously, wires 547 are isolated and separated to ensure patient safety. Cannula 512 may have separate lumens to pass electricity, and these lumens are also equipped with appropriate safety mechanisms.

In operation, the surgeon or nurse connects the cameras 532 to the system controller 556 prior to inserting the cannula system 510. Video wire 547 runs through the separate electricity lumens. Cameras 532 are activated when connected and start transmitting video images to the system. Thus, the surgeon can view the video stream and use it during cannula insertion to make insertion easier. Obturator 516 is inserted through the proximal end 526 of the cannula 512, through the rear portion 515 and disposed within the central lumen 508 of cannula 512 so it extends through the body 515 so the distal end 548 of the obturator extends out the distal end 528 of the cannula.

The surgeon or nurse then inserts the cannula 512 together with the obturator 516 into the surgical site of the patient. The surgeon may use the cutting edge 520 of the obturator 516 to cut tissue in the surgical site as needed and may connect the irrigation tube 576 as needed. Once the cannula system 510 is positioned deeply enough in the surgical site and the surgeon is finished with the obturator 516, he or she withdraws it from the central lumen 508 of cannula system 510 by pulling it out of cannula 512 via its distal end 528. At this point, when cannula system 510 is fully placed, the surgeon can start using the video feed for the procedure itself right away. After completing the procedure, the surgeon or nurse retracts the cannula system 510 from the surgical site.

FIG. 34 shows an exemplary internal structure of a computer 1250 in which various embodiments of the present disclosure may be implemented. Computer 1250 contains a system bus 1279, where a bus is a set of hardware lines used for data transfer among the components of a computer or processing system. Bus 1279 is essentially a shared conduit that connects different elements of a computer system (e.g., processor, disk storage, memory, input/output ports, network ports, etc.) that enables the transfer of information between the elements. Attached to system bus 1279 is I/O device interface 1282 for connecting various input and output devices (e.g., sensors, transducers, keyboard, mouse, displays, printers, speakers, etc.) to the computer 1250. Network interface 1286 allows the computer 1250 to connect to various other devices attached to a network.

Memory 1090 provides volatile storage for computer software instructions 1292 and data 1294 used to implement embodiments of the present disclosure. Disk storage 1295 provides non-volatile storage for computer software instructions 1292 and data 1294 used to implement an embodiment of the present disclosure. Central processor unit 1284 is also attached to system bus 1279 and provides for the execution of computer instructions.

In an exemplary embodiment, the processor routines 1292 (e.g., instructions for the processes/calculations described above) and data 1094 are a computer program product (generally referenced 1292), including a computer readable medium (e.g., a removable storage medium such as one or more DVD-ROMs, CD-ROMs, diskettes, tapes, etc.) that provides at least a portion of the software instructions for the invention system. Computer program product 1292 can be installed by any suitable software installation procedure, as is well known in the art. In another embodiment, at least a portion of the software instructions may also be downloaded over a cable, communication and/or wireless connection. Further, the present embodiments may be implemented in a variety of computer architectures. The computer of FIG. 36 is for purposes of illustration and not limitation of the present disclosure.

Thus, it is seen that improved and associated cannula systems and methods of providing 3D images for surgical procedures are provided. It should be understood that any of the foregoing configurations and specialized components or chemical compounds may be interchangeably used with any of the systems of the preceding embodiments. Although illustrative embodiments are described hereinabove, it will be evident to one skilled in the art that various changes and modifications may be made therein without departing from the disclosure. It is intended in the appended claims to cover all such changes and modifications that fall within the true spirit and scope of the disclosure.

While the disclosed systems and devices have been described in terms of what are presently considered to be the most practical exemplary embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A cannula system comprising:
at least one cannula having a proximal end, a distal end, a central lumen, and an insertion axis;
a disc mounted to the distal end of the cannula, the disc comprising two separable half portions;
a camera and one or more lighting modules housed in each of the separable half portions of the disc, each camera being aligned with the insertion axis; and
an obturator configured to be at least partially disposed within the at least one cannula, the obturator having a proximal end and a distal end;
wherein when the obturator is removed the two separable half portions of the disc can be slid apart.

2. The cannula system of claim 1 wherein each separable half portion of the disc has a flange defining at least one internal slot, and a camera is housed in each internal slot.

3. The cannula system of claim 1 wherein the disc defines a channel configured to house one or more wires.

4. The cannula system of claim 1 wherein the at least one cannula defines at least one inner lumen.

5. The cannula system of claim 1 wherein each camera streams live video during insertion of the cannula system.

6. The cannula system of claim 1 further comprising a processor in communication with each camera.

7. The cannula system of claim 6 wherein the processor receives images from the camera and provides the images in three dimensions.

8. The cannula system of claim 1 further comprising two handles configured to slide apart the two separable half portions of the disc.

9. The cannula system of claim 1 wherein the obturator is substantially flat with a cone-shaped distal end having a cutting edge.

10. The cannula system of claim 9 wherein the cone-shaped distal end defines at least one channel configured to be aligned with each camera such that each camera has an unobstructed view of a surgical site along the insertion axis.

11. A method of providing under vision insertion and 3D imaging for surgery, comprising:
providing at least one cannula having a proximal end, a distal end, a central lumen, an insertion axis, and a disc mounted to the distal end of the cannula, the disc comprising two separable half portions;
housing a camera and one or more lighting modules in each of the separable half portions of the disc such that each camera is aligned with the insertion axis;
inserting the at least one cannula and an obturator partially disposed within the central lumen of the at least one cannula into a surgical joint or cavity space;
removing the obturator from the cannula; and
sliding the two separable half portions of the disc apart.

12. The method of claim 11 wherein sliding the two separable half portions of the disc apart comprises rotating one or more handles.

13. The method of claim 11 wherein each camera starts streaming live video prior to the insertion step.

14. The method of claim 11 wherein the one or more lighting modules are LEDs.

15. The method of claim 11 further comprising providing a processor in communication with each camera.

16. The method of claim 11 wherein the obturator is substantially flat with a cone-shaped distal end.

17. The method of claim 16 wherein the cone-shaped distal end of the obturator defines at least one channel configured to be aligned with each camera such that each camera has an unobstructed view of a surgical site along the insertion axis.

18. The method of claim 11 wherein each separable half portion of the disc has a flange defining at least one internal slot, and a camera is housed in each internal slot.

19. The method of claim 11 wherein the disc defines a channel configured to house one or more wires.

20. The method of claim 11 further comprising removing the cannula from the surgical site.
